(19) United States
(12) Patent Application Publication
(10) Pub. No.: US 2012/0010176 A1
(43) Pub. Date: Jan. 12, 2012

(54) UTROPHIN PROMOTER ACTIVITY UPREGULATION FOR THE TREATMENT OF MUSCULAR DYSTROPHY

(76) Inventors: Tejvir S. Khurana, Narnerth, PA (US); Scott L. Diamond, Bala Cynwyd, PA (US); Catherine Moorwood, Philadelphia, PA (US); Olga Lozynska, Philadelphia, PA (US); Andrew D. Napper, Chadds Ford, PA (US); Emidio Pistilli, Deptford, NJ (US); Neha Suri, Philadelphia, PA (US)

(21) Appl. No.: 13/129,074

(22) PCT Filed: Nov. 12, 2009
(86) PCT No.: PCT/US09/64230

§ 371 Date: Oct. 3, 2011

Related U.S. Application Data
(60) Provisional application No. 61/114,018, filed on Nov. 12, 2008.

(51) Int. Cl. A61K 31/352; A61K 31/4525; A61K 31/12; A61K 31/428; A61K 31/05; A61P 21/00; A61K 31/427; A61K 31/42; A61K 31/351; A61K 31/138; A61K 31/565; C12N 5/071; A61K 31/655

(52) U.S. Cl. 514/150; 435/375; 514/321; 514/456; 514/682; 514/367; 514/734; 514/365; 514/378; 514/460; 514/651; 514/178

(57) ABSTRACT

Methods are provided for increasing utrophin promoter activity, utrophin expression or utrophin activity, and treating or reducing the symptoms of muscular dystrophy by administering to a subject in need thereof an effective amount of a composition comprising a utrophin promoter or activity upregulator. Muscular dystrophy includes but is not limited to Duchenne muscular dystrophy, Becker's muscular dystrophy, limb girdle muscular dystrophy, or a disease characterized by mutation or dysregulation of the dystrophin gene or an aberrant or dysfunctional dystrophin.

### CROSS-REFERENCE TO RELATED APPLICATIONS

This application claims the benefit of priority from U.S. Provisional Applications 61/114,018 and 61/116,013, filed Nov. 12, 2008 and Nov. 19, 2008, respectively, both of which are incorporated by reference herein in their entirety.

### FIELD OF THE INVENTION

This invention provides compounds and pharmaceutical compositions thereof that upregulate utrophin promoter activity and methods of use thereof. Such compounds can be useful for treating or reducing the symptoms of diseases such as muscular dystrophy in particular those involving dysregulation or dysfunction of the dystrophin gene or gene product.

### BACKGROUND OF THE INVENTION

Duchenne muscular dystrophy (DMD) is one of a group of muscular dystrophies characterized by the enlargement of muscles. DMD is one of the most prevalent types of muscular dystrophy and is characterized by rapid progression of muscle degeneration which occurs early in life. DMD is X-linked and affect mainly males—an estimated 1 in 3,500 boys worldwide. The degenerative muscle wasting and weakness usually culminates in lethality by age 30 to 40. While the etiology and pathogenesis of the disease are well defined, DMD remains an incurable affliction.

The gene for DMD, found on the X chromosome, encodes a large protein-dystrophin. Dystrophin is required inside muscle cells for structural support: it is thought to strengthen muscle cells by anchoring elements of the internal cytoskeleton to the surface membrane and external structures. Without it, the muscle cannot produce force effectively and is susceptible to damage during contraction, eventually leading to muscle death and replacement by fatty and fibrous tissue. The accompanying immune response can add to the damage.

In addition to DMD, other diseases referred to as muscular dystrophy share a similar pathophysiology related to mutation or dysregulation of the dystrophin gene or an aberrant or dysfunctional dystrophin protein. Such other forms of muscular dystrophy include Becker's muscular dystrophy and some forms of limb girdle muscular dystrophy.

Utrophin is an autosomal (6q24 encoded) homolog of dystrophin. In vivo, utrophin upregulation in the mdx mouse model of DMD (achieved via transgenic, viral vector, and transcription factor perturbation approaches) physiologically substitutes for dystrophin deficiency and can correct the disease.

### SUMMARY OF THE INVENTION

In one embodiment, a method is provided for treating muscular dystrophy comprising administering an effective amount of a small molecule compound. In another embodiment the small molecule compound is administered orally or parenterally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating muscular dystrophy comprising administering an effective amount of a composition comprising a compound of formula (I) below:

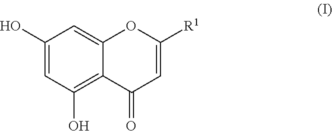

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms of muscular dystrophy comprising administering an effective amount of a small molecule compound. In another embodiment the small molecule compound is administered orally or parenterally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms of muscular dystrophy comprising administering an effective amount of a composition comprising a compound of formula (I) below:

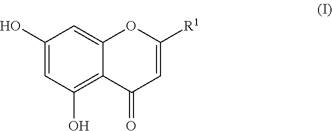

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In one embodiment, a method is provided for increasing activity of the utrophin promoter in a subject by administering to the subject an effective amount of a composition comprising a small molecule compound. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In one embodiment, a method is provided for increasing activity of the utrophin promoter in a subject comprising administering an effective amount of a composition comprising a compound of formula (I):

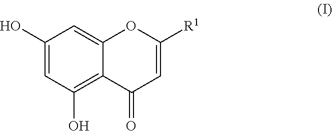

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In one embodiment, a method is provided for increasing expression or activity of utrophin in a subject by administering to the subject an effective amount of a composition comprising a small molecule compound. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In one embodiment, a method is provided for increasing expression or activity of utrophin in a subject by administering to the subject an effective amount of a composition comprising a compound of formula (I) below:

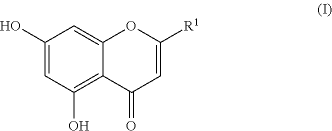

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for correcting a deficiency of dystrophin in a subject by administering to the subject an effective amount of a composition comprising a small molecule compound. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for correcting a deficiency of dystrophin in a subject comprising administering to the subject an effective amount of a composition comprising a compound of formula (I) below:

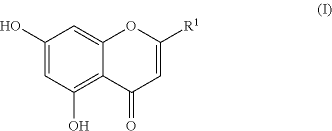

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating a subject with muscular dystrophy by administering an effective amount of a composition comprising a small molecule compound that increases activity of the utrophin promoter. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating a subject with muscular dystrophy comprising administering to the subject an effective amount of a composition comprising a compound of formula (I) below:

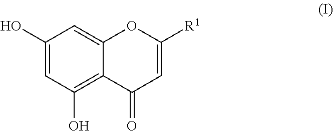

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating a subject with muscular dystrophy by administering an effective amount of a composition comprising a small molecule compound that increases expression or activity of utrophin. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating a subject with muscular dystrophy comprising administering to the subject an effective amount of a composition comprising a compound of formula (I) below:

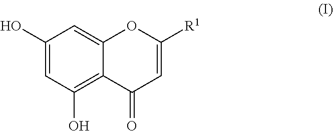

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms associated with muscular dystrophy in a subject comprising administering an effective amount of a composition comprising a small molecule compound that increases activity of the utrophin promoter. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms associated with muscular dystrophy in a subject comprising administering to the subject an effective amount of a composition comprising a compound of formula (I) below,

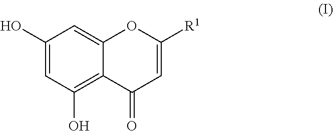

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms associated with muscular dystrophy in a subject comprising administering an effective amount of a composition comprising a small molecule compound that increases expression or activity of utrophin. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms associated with muscular dystrophy comprising administering to the subject an effective amount of a composition comprising a compound of formula (I) below:

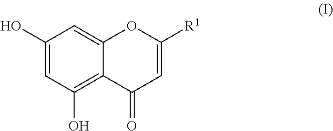

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating muscular dystrophy comprising the step of administering an effective amount of a small molecule compound, wherein muscular dystrophy is treated. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating muscular dystrophy comprising the step of administering an effective amount of a compound of formula (I) below:

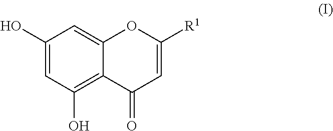

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing, wherein muscular dystrophy is treated.

In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms of muscular dystrophy comprising the step of administering an effective amount of a small molecule compound, wherein the symptoms of muscular dystrophy are reduced. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms of muscular dystrophy comprising the step of administering an effective amount of a compound of formula (I) below:

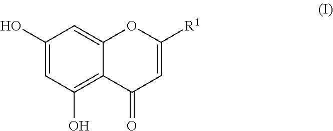

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing,
   wherein the symptoms of muscular dystrophy are reduced.

In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In one embodiment, a method is provided for increasing activity of the utrophin promoter in a subject comprising the step of administering to the subject an effective amount of a composition comprising a small molecule compound, whereby activity of the utrophin promoter is increased. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In one embodiment, a method is provided for increasing activity of the utrophin promoter in a subject comprising the step of administering to the subject an effective amount of a composition comprising a compound of formula (I) below:

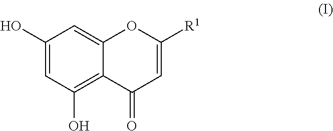

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing,
   whereby activity of the utrophin promoter is increased.

In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In one embodiment, a method is provided for increasing expression or activity of utrophin in a subject comprising the step of administering to the subject an effective amount of a composition comprising a small molecule compound, wherein the expression or activity of utrophin is increased. In another embodiment, the small molecule compound is administered parenterally or orally. In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In one embodiment, a method is provided for increasing expression or activity of utrophin in a subject comprising the step of administering to the subject an effective amount of a composition comprising a compound of formula (I) below:

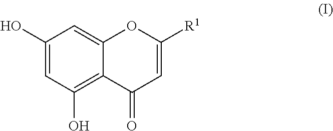

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing,
   wherein the expression or activity of utrophin is increased.

In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for correcting a deficiency of dystrophin in a subject comprising the step of by administering to the subject an effective amount of a composition comprising a small molecule compound, wherein the deficiency of dystrophin is improved or corrected. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for correcting a deficiency of dystrophin in a subject comprising the step of by administering to the subject an effective amount of a composition comprising a compound of formula (I) below:

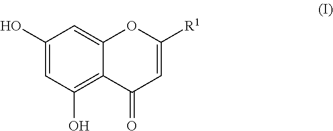

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing, wherein the deficiency of dystrophin is improved or corrected.

In another embodiment, the subject suffers from muscular dystrophy. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating a subject with muscular dystrophy comprising the step of administering an effective amount of a composition comprising a small molecule compound that increases activity of the utrophin promoter, wherein muscular dystrophy is treated. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating a subject with muscular dystrophy comprising the step of administering an effective amount of a composition comprising a a compound of formula (I) below:

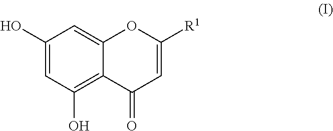

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing, that increases activity of the utrophin promoter,
   wherein muscular dystrophy is treated. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating a subject with muscular dystrophy comprising the step of by administering an effective amount of a composition comprising a small molecule compound that increases expression or activity of utrophin, wherein muscular dystrophy is treated. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for treating a subject with muscular dystrophy comprising the step of by administering an effective amount of a composition comprising a compound of formula (I) below:

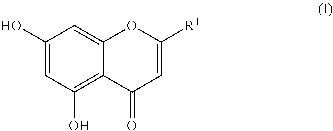

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing, that increases expression or activity of utrophin,
   wherein muscular dystrophy is treated. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms associated with muscular dystrophy in a subject comprising the step of administering an effective amount of a composition comprising a small molecule compound that increases activity of the utrophin promoter, wherein the symptoms of muscular dystrophy are reduced. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms associated with muscular dystrophy in a subject comprising the step of administering an effective amount of a composition comprising a compound of formula (I) below:

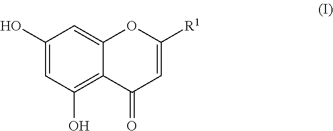

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing,
   that increases activity of the utrophin promoter, wherein the symptoms of muscular dystrophy are reduced. In another embodiment, the small molecule compound is administered parenterally or orally. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms associated with muscular dystrophy in a subject comprising the step of administering an effective amount of a composition comprising a small molecule compound that increases expression or activity of utrophin, wherein the symptoms of muscular dystrophy are reduced. In another embodiment, the small molecule compound is administered parenterally or orally. In another embodiment, the small molecule compound is piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for reducing the symptoms associated with muscular dystrophy in a subject comprising the step of administering an effective amount of a composition comprising a compound of formula (I) below:

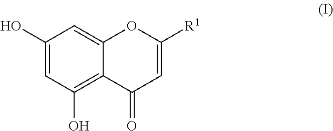

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing,
   that increases expression or activity of utrophin, wherein the symptoms of muscular dystrophy are reduced. In a further embodiment, muscular dystrophy is Duchenne muscular dystrophy, Becker's muscular dystrophy, limb girdle muscular dystrophy, or a disease characterized by mutation or dysregulation of the dystrophin gene or an aberrant or dysfunctional gene product.

In another embodiment, a method is provided for upregulating utrophin expression or activity in a cell comprising exposing the cell to an effective amount of a compound selected from among piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing.

In another embodiment, a method is provided for upregulating utrophin expression in a cell comprising exposing the cell to an effective amount of a compound of formula (I) below:

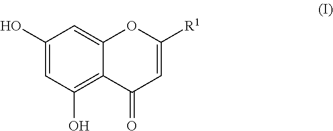

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
   or an isomer, prodrug, active metabolite, analog, or a pharmaceutically-acceptable derivative or salt form of any of the foregoing.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

FIG. 1 shows dose response curves for the compounds described herein in 384-well format;

FIG. 2 shows dose response curves for the compounds described herein in 96-well format;

FIG. 3 shows upregulation of utrophin A mRNA after treatment of C2C12 muscle cells with certain compounds described herein.

FIG. 4 shows that compounds can increase endogenous Utrophin A mRNA. C2C12 cells were treated with Prestwick compounds A, J, L and S plus TSA and heregulin (HRG) as positive controls. All compounds increased levels of endogenous utrophin A mRNA either in serum-containing or serum-free media.

FIG. 5 shows that muscle weights are increased on treatment. Soleus, gastrocnemius (gastroc), tibialis anterior (TA) and quadriceps (quads) were bigger in treated mice. EDL was not significantly bigger (* p<0.05). There was no difference in whole body weight between treatment groups.

FIG. 6 shows that treated muscles show increased force generation. Muscle function tested using a variety of parameters showed a trend towards increased force generating capacity and decreased force drop on eccentric contraction. Peak specific twitch force was significantly increased (* p<0.05).

### DEFINITIONS

It is understood that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic, carbon and heteroatom substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment and prevention, for example of disorders, as described generally above. Examples of substituents include, but are not limited to aliphatic; heteroaliphatic; alicyclic; heterocyclic; aromatic, heteroaromatic; aryl; heteroaryl; alkylaryl; aralkyl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; or —GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heterocyclic, aromatic, heteroaromatic, aryl, heteroaryl, alkylaryl, or alkylheteroaryl moiety.

The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched) or branched aliphatic hydrocarbons as defined by IUPAC, which are optionally substituted with one or more functional groups. As defined herein, “aliphatic” is intended to include optionally substituted alkyl, alkenyl and alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having about 1-6 carbon atoms. In some instances aliphatic can include alicyclic or cycloalkyl, including unsaturations therein.

In certain embodiments, the alkyl, alkenyl and alkynyl groups employed in the invention contain 1-20; 2-20; 3-20; 4-20; 5-20; 6-20; 7-20 or 8-20 aliphatic carbon atoms. In certain other embodiments, the alkyl, alkenyl, and alkynyl groups employed in the invention contain 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl, alkenyl, and alkynyl groups employed in the invention contain 1-6; 2-6; 3-6; 4-6 or 5-6 aliphatic carbon atoms. In yet other embodiments, the alkyl, alkenyl, and alkynyl groups employed in the invention contain 1-4; 2-4 or 3-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargy 1), 1-propynyl and the like.

The term “alicyclic”, as used herein, refers to compounds that combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl-n, cyclohexyl, —CH₂-cyclohexyl, cyclohexenylethyl, cyclohexanylethyl, norborbyl moieties and the like, which again, may bear one or more substituents.

The term “cycloalkyl”, as used herein, refers to cyclic alkyl groups, specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, heteroaliphatic or heterocyclic moieties, may optionally be substituted. An analogous convention applies to other generic terms such as “cycloalkenyl”, “cycloalkynyl” and the like.

The term “heteroaliphatic”, as used herein, refers to aliphatic moieties in which one or more carbon atoms in the main chain have been replaced with a heteroatom. Thus, a heteroaliphatic group refers to an aliphatic chain which contains one or more oxygen, sulfur, nitrogen, phosphorus or silicon atoms in place of carbon atoms in the aliphatic main chain. Heteroaliphatic moieties may be branched or linear unbranched. In certain embodiments, heteroaliphatic moieties are substituted by independent replacement of one or more of the hydrogen atoms thereon with one or more moieties including, but not limited to aliphatic; heteroaliphatic; alicyclic; heterocyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; or —GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heterocyclic, aromatic, heteroaromatic, aryl, heteroaryl, alkylaryl, or alkylheteroaryl moiety.

The term “heteroalicyclic”, “heterocycloalkyl” or “heterocyclic”, as used herein, refers to compounds which combine the properties of heteroaliphatic and cyclic compounds and include but are not limited to saturated and unsaturated mono- or polycyclic ring systems having 5-16 atoms wherein at least one ring atom is a heteroatom selected from O, S and N (wherein the nitrogen and sulfur heteroatoms may be optionally be oxidized), wherein the ring systems are optionally substituted with one or more functional groups, as defined herein. In certain embodiments, the term “heterocyclic” refers to a non-aromatic 5-, 6- or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 2 double bonds and each 6-membered ring has 0 to 2 double bonds, (ii) the nitrogen and sulfur heteroatoms may optionally be oxidized, (iii) the nitrogen heteroatom may optionally be quaternized, and (iv) any of the above heterocyclic rings may be fused to an aryl or heteroaryl ring. Representative heterocycles include, but are not limited to, pyrrolidinyl, pyrazolinyl, pyrazolidinyl imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one or more hydrogen atoms thereon with aliphatic; heteroaliphatic; alicyclic; heterocyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; or —GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heterocyclic, aromatic, heteroaromatic, aryl, heteroaryl, alkylaryl, or alkylheteroaryl moiety.

Additionally, it will be appreciated that any of the alicyclic or heterocyclic moieties described above and herein may comprise an aryl or heteroaryl moiety fused thereto.

In general, the term “aromatic moiety”, as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”. Examples of aromatic moieties include, but are not limited to, phenyl, indanyl, indenyl, naphthyl, phenanthryl and anthracyl.

In general, the term “heteroaromatic moiety”, as used herein, refers to stable substituted or unsubstituted unsaturated mono-heterocyclic or polyheterocyclic moieties having preferably 3-14 carbon atoms, comprising at least one ring having p-orbitals perpendicular to the plane of the ring at each ring atom, and satisfying the Huckel rule where the number of pi electrons in the ring is (4n+2) wherein n is an integer. Examples of heteroaromatic moieties include, but are not limited to, pyridyl, quinolinyl, dihydroquinolinyl, isoquinolinyl, quinazolinyl, dihydroquinazolyl, and tetrahydroquinazolyl.

It will also be appreciated that aromatic and heteroaromatic moieties, as defined herein, may be attached via an aliphatic (e.g., alkyl) or heteroaliphatic (e.g., heteroalkyl) moiety and thus also include moieties such as -(aliphatic)aromatic, -(heteroaliphatic)aromatic, -(aliphatic)heteroaromatic, -(heteroaliphatic)heteroaromatic, -(alkyl)aromatic, -(heteroalkyl)aromatic, -(alkyl)heteroaromatic, and -(heteroalkyl)heteroaromatic moieties. Thus, as used herein, the phrases “aromatic or heteroaromatic moieties” and “aromatic, heteroaromatic, ⁻(alkyl)aromatic, -(hetero alkyl) aromatic, -(hetero alkyl)hetero aromatic, and -(heteroalkyl)heteroaromatic” are interchangeable. In some instances corresponding moieties may be referred to synonymously as aralkyl, heteroaralkyl and the like. Substituents include, but are not limited to, any of the previously mentioned substituents, i.e., the substituents recited for aliphatic moieties, or for other moieties as disclosed herein, resulting in the formation of a stable compound.

In general, the term “aryl” refers to aromatic moieties, as described above, excluding those attached via an aliphatic (e.g., alkyl) or heteroaliphatic (e.g., heteroalkyl) moiety. In certain embodiments of the present invention, “aryl” refers to a mono- or bicyclic carbocyclic ring system having one or two rings satisfying the Huckel rule for aromaticity, including, but not limited to, phenyl, naphthyl, tetrahydronaphthyl, indanyl, indenyl and the like.

Similarly, the term “heteroaryl” refers to heteroaromatic moieties, as described above, excluding those attached via an aliphatic (e.g., alkyl) or heteroaliphatic (e.g., heteroalkyl) moiety. In certain embodiments of the present invention, the term “heteroaryl”, as used herein, refers to a cyclic unsaturated radical having from about five to about ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

As defined herein, “aryl” and “heteroaryl” groups (including bicyclic aryl groups) can be unsubstituted or substituted, wherein substitution includes replacement of one or more of the hydrogen atoms thereon independently with any of the previously mentioned substitutents, i.e., the substituents recited for aliphatic moieties, or for other moieties as disclosed herein, resulting in the formation of a stable compound. For example, aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; heteroaliphatic; alicyclic; heterocyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; or —GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, OC(═O)O—, —OC(═O)NR^{G2}—, NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heterocyclic, aromatic, heteroaromatic, aryl, heteroaryl, alkylaryl, or alkylheteroaryl moiety. Additionally, it will be appreciated, that any two adjacent groups taken together may represent a 4, 5, 6, or 7-membered substituted or unsubstituted alicyclic or heterocyclic moiety.

The term “alkoxy” or “alkyloxy”, as used herein refers to a saturated (i.e., O-alkyl) or unsaturated (i.e., O-alkenyl and O-alkynyl) group attached to the parent molecular moiety through an oxygen atom. In certain embodiments, the alkyl group contains 1-20; 2-20; 3-20; 4-20; 5-20; 6-20; 7-20 or 8-20 aliphatic carbon atoms. In certain other embodiments, the alkyl group contains 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl group contains 1-6; 2-6; 3-6; 4-6 or 5-6 aliphatic carbon atoms. In yet other embodiments, the alkyl group contains 1-4; 2-4 or 3-4 aliphatic carbon atoms. Examples of alkoxy, include but are not limited to, methoxy, ethoxy, propoxy, isopropoxy, n-butoxy, i-butoxy, sec-butoxy, tert-butoxy, neopentoxy, n-hexoxy and the like.

The term “thioalkyl” as used herein refers to a saturated (i.e., S-alkyl) or unsaturated (i.e., S-alkenyl and S-alkynyl) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of thioalkyl include, but are not limited to, methylthio, ethylthio, propylthio, isopropylthio, n-butylthio, and the like.

The term “alkylamino” refers to a group having the structure —NHR′ wherein R′ is aliphatic or alicyclic, as defined herein. The term “aminoalkyl” refers to a group having the structure NH₂R′—, wherein R′ is aliphatic or alicyclic, as defined herein. In certain embodiments, the aliphatic or alicyclic group contains 1-20 aliphatic carbon atoms. In certain other embodiments, the aliphatic or alicyclic group contains 1-10 aliphatic carbon atoms. In still other embodiments, the aliphatic or alicyclic group contains 1-6 aliphatic carbon atoms. In yet other embodiments, the aliphatic or alicyclic group contains 1-4 aliphatic carbon atoms. In yet other embodiments, R′ is an alkyl, alkenyl, or alkynyl group containing 1-8 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aryl or heteroaryl substituents described above and herein may be substituted or unsubstituted.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}) or quaternary (—N⁺RₓR_{y}R_{z}) amine, where Rₓ, R_{y} and R_{z} are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “acyl”, as used herein, refers to a group having the general formula —C(═O)R, where R is an aliphatic, alicyclic, heteroaliphatic, heterocyclic, aromatic or heteroaromatic moiety, as defined herein.

The term “C₂₋₆alkenylene”, as used herein, refers to a substituted or unsubstituted, linear or branched unsaturated divalent radical consisting solely of carbon and hydrogen atoms, having from two to six carbon atoms, having a free valence “—” at both ends of the radical, and wherein the unsaturation is present only as double bonds and wherein a double bond can exist between the first carbon of the chain and the rest of the molecule.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like encompass substituted and unsubstituted, and saturated and unsaturated groups. Additionally, the terms “cycloalkyl”, “cycloalkenyl”, “cycloalkynyl”, “heterocycloalkyl”, “heterocycloalkenyl”, “heterocycloalkynyl”, “aromatic”, “heteroaromatic”, “aryl”, “heteroaryl” and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Another example is an N-methyl derivative of a compound, which is susceptible to oxidative metabolism resulting in N-demethylation. Pro-drugs of a variety of compounds, and materials and methods for derivatizing the parent compounds to create the pro-drugs, are known and may be adapted to the present invention.

### DETAILED DESCRIPTION OF THE INVENTION

In various embodiments disclosed herein, methods are provided for addressing the symptoms and the disease known as muscular dystrophy using small molecule therapy. In one embodiment, small molecule therapy alleviates the symptoms of the disease. In another embodiment small molecule therapy increases activity of the utrophin promoter or increases expression or activity of utrophin, which corrects a deficiency in dystrophin that characterizes the disease. In another embodiment, small molecule therapy improves walking of a muscular dystrophy patient. In another embodiment, the methods described herein reduce or inhibit swelling of the calves with fibrous tissue. In another embodiment, the methods described herein induce muscle growth. In another embodiment, the methods described herein induce muscle regeneration. In another embodiment, the methods described herein reduce or inhibit contractures. In another embodiment, the methods described herein reduce or inhibit scoliosis. In another embodiment, the methods described herein reduce or inhibit diaphragm weakening.

Muscular dystrophy includes but is not limited to diseases in which the dystrophin gene is mutated or dysregulated, or the gene product, dystrophin, is aberrant or dysfunctional. Such forms of muscular dystrophy include but are not limited to Duchenne muscular dystrophy, Becker's muscular dystrophy, or limb girdle muscular dystrophy. Furthermore, muscular dystrophy comprises any other dystrophy or disease characterized by mutation or dysregulation of the dystrophin gene or an aberrant or dysfunctional gene product.

In another embodiment, the small molecule compound useful for any of the above embodiments has the structure (I) below,

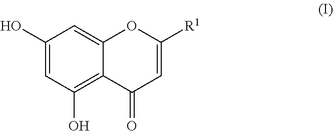

wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl.

In certain embodiments, R¹ phenyl or a substituted phenyl. In other embodiments, the substituted phenyl is 4-hydroxyphenyl or 3-hydroxy-4-methoxyphenyl.

In another embodiment, the composition comprising the small molecule compound useful for any of the above embodiments is 1-[5-(1,3-benzodioxol-5-yl)-1-oxo-2,4-pentadienyl]piperidine, compound (II) below, commonly known as piperine.

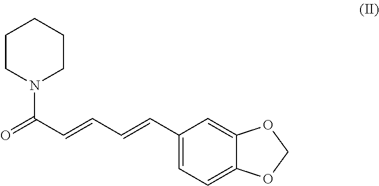

Piperine is the alkaloid responsible for the pungency of black pepper and long pepper, along with chavicine (an isomer of piperine, (2Z,4Z)-5-(1,3-benzodioxol-5-yl)-1-(1-piperidyl)penta-2,4-dien-1-one). In addition to chavicine, other piperine analogs useful for the purposes herein include 1-(3,4-methylenedioxyphenyl)-penta-2E,4E-dienoic acid methyl ester, 1-E,E-piperinoyl-isobutylamine and 1-(3,4-methylenedioxyphenyl)-pentanoic acid cyclohexyl amide.

In another embodiment, the small molecule compound useful for any of the above embodiments is 5,7-dihydroxy-2-(4-hydroxyphenyl)-4H-1-benzopyran-4-one, compound (III) below, commonly known as apigenin.

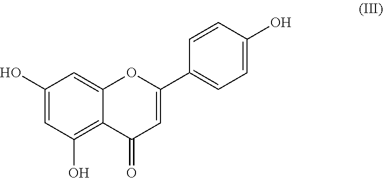

Apigenin is a flavone that is the aglycone of apiin, isolated from parsley and celery, and apigetrin. It is a yellow crystalline solid that has been used to dye wool.

Apigenin analogs also embodied herein include ethyl-7-O-apigenin-glucuronate, apigenin-7-O-beta-glucoside, and apigenin-7-O-neohesperidoside. Other analogs include acacetin, chrysin, kampherol, luteolin, myricetin, naringenin, and quercetin

In another embodiment, the small molecule compound useful for any of the above embodiments is 5,7-dihydroxy-2-phenyl-4H-chromen-4-one, having the structure (IV) below and commonly known as chrysin.

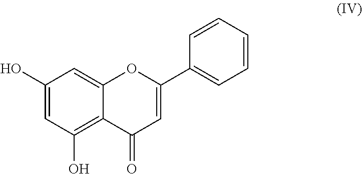

Chrysin is a naturally occurring flavone chemically extracted from the blue passionflower (Passiflora caerulea). Honeycomb also contains small amounts. Other names for chrysin are 5,7-dihydroxyflavone, 5,7-dihydroxy-2-phenyl-(9CI), NP-005901, and galangin flavanone. Chrysin analogs embodied herein include quercetin, apigenin, luteolin and diosmetin.

In another embodiment, the small molecule compound useful for any of the above embodiments is 4-(6-methoxy-2-naphthyl)-2-butanone, compound (V) below, commonly known as nabumetone.

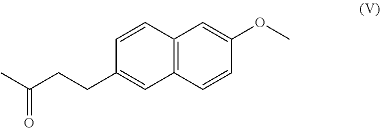

Nabumetone is a non-steroidal anti-inflammatory drug of the arylalkanoic acid family (which includes diclofenac). It is manufactured by Meda, and is available under the brand names Relafen, Relifex and Gambaran.

Nabumetone analogs include the active metabolite 6-methoxy-2-naphthylacetic acid.

In another embodiment, the small molecule compound useful for any of the above embodiments is 6-(trifluoromethoxy)benzothiazol-2-amine, compound (VI) below, commonly known as riluzole. Riluzole is a drug used to treat amyotrophic lateral sclerosis. It delays the onset of ventilator-dependence or tracheostomy in selected patients and may increase survival by approximately two months.

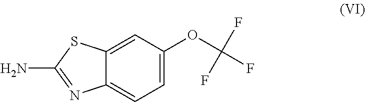

Riluzole analogs embodied herein include 2-imino-3-(2-methylthio)-ethyl-6-trifluoromethoxybenzothiazoline and 2-imino-3-(2-methylsulfinyl)-ethyl-6-trifluoromethoxybenzothiazoline.

In another embodiment, the small molecule compound useful for any of the above embodiments is (S)-2,3-dihydro-5,7-dihydroxy-2-(3-hydroxy-4-methoxyphenyl)-4H-1-benzopyran-4-one, compound (VII) below, commonly known as hesperetin. Hesperetin is a bioflavonoid and, to be more specific, a flavanone. It is the aglycone of hesperidin, found in citrus fruits.

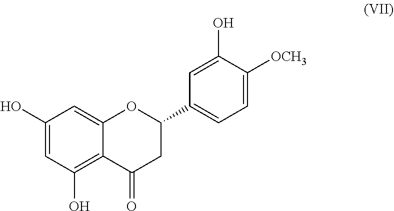

Hesperetin analogs include hesperin ((2S)-5,7-dihydroxy-2-(3-hydroxy-4-methoxyphenyl)chroman-4-one) and hesperedin (5-Hydroxy-2-(3-hydroxy-4-methoxyphenyl)-4-oxo-4H-chromen-7-yl rutinoside).

In another embodiment, the small molecule compound useful for any of the above embodiments is trans-3,4′,5-trihydroxystilbene, compound (VIII) below, commonly known as resveratrol. Resveratrol is a phytoalexin produced naturally by several plants when under attack by pathogens such as bacteria or fungi. Resveratrol is also found in the skin of red grapes and is a constituent of red wine.

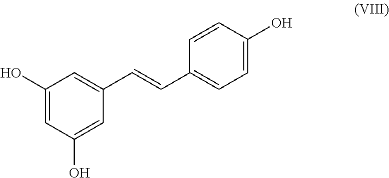

Synonyms for resveratrol include 3,4′,5-stilbenetriol; trans-resveratrol; and (E)-5-(p-hydroxystyryl)resorcinol. Analogs embodied herein include 3,5-dihydroxystilbene, 3,3′,4,5′-tetrahydroxystilbene, 3,4,4′,5-tetrahydroxystilbene, 3,3′,5,5′-tetrahydroxystilbene, 3,3′,4,5,5′-pentahydroxystilbene, 3,5-dimethoxystilbene, 3,4′,5-trimethoxystilbene, 3,3′,4,5′-tetramethoxystilbene, 3,4,4′,5-tetramethoxystilbene, 3,3′,5′5′-tetramethoxystilbene, and 3,3′,4,5,5′-pentamethoxystilbene.

In another embodiment, the small molecule compound useful for any of the above embodiments is 3-phenyldiazenylpyridine-2,6-diamine, compound (IX) below, commonly known as phenazopyridine. Phenazopyridine is a chemical which, when secreted into the urine, has a specific local analgesic effect. It is often used to alleviate the pain, irritation, discomfort, or urgency caused by urinary tract infections, surgery, or injury to the urinary tract.

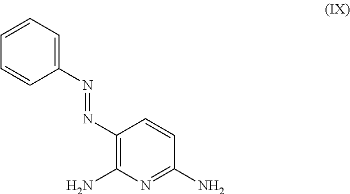

Phenazopyridine analogs embodied herein include 4′-N,N-dimethylamino-1′-phenylazo-3-pyridine, 4′-N,N-diethylamino-1′ phenylazo-3-pyridine; 4′-N,N-di-(beta-hydroxyethylamino)-1′ phenylazo-3-pyridine; 4′-N-methylamino sulfonic acid-1′-phenylazo-3-pyridine; 4′-N,N-dimethylamino-6′-acetamido-1′ phenylazo-3-pyridine, and 4′-N,N-di-(beta-hydroxyethylamino)-6′-methyl-1′phenylazo-3-pyridine. An active metabolite embodied herein is 4′-N,N-dimethylamino-6-methoxy-phenylazo-3-pyridine.

In another embodiment, the small molecule compound useful for any of the above embodiments is 2-(1,3-thiazol-4-yl)-1H-benzoimidazole, compound (X) below, commonly known as tiabendazole. Thiabendazole is a fungicide and parasiticide. It is known by the brand names Mintezo and Arbotect among others.

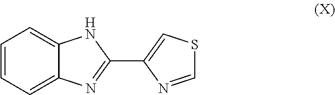

Tiabendazole analogs and brand names embodied herein include Apl-Luster; Arbotect; Bioguard; Bovizole; Chemviron TK 100; Cropasal; Drawipas; Eprofil; Equivet TZ; Equizole; Hokustar hp; Hymush; Lombristop; Mertec; Mertect; Mertect 160; Mertect 340f; Mertect lsp; Metasol TK 100; Metasol TK 10; Mintesol; Mintezol; Minzolum; Mycozol; Nemacin; Nemapan; Omnizole; Ormogal; Polival; RPH; Sanaizol 100; Sistesan; Storite; TBZ 6; TBZ 60W; TBDZ; TBZ; Tebuzate; Tecto; Tecto 10P; Tecto 40F; Tecto 60; Tecto B; Tecto rph; Testo; Thiaben; Thiabendazol; Thiabendole; Thiabenzazole; Thiabenzole; Thibendole; Thibenzol; Thibenzole; Thibenzole 200; Thibenzole att; Thiprazole; Tiabenda; Tocamide; Tocamidee; Tibimix 20; Tobaz; Top form wormer; Triasox; and Tubazole.

In another embodiment, the small molecule compound useful for any of the above embodiments is 5-methyl-N-[4-(trifluoromethyl)phenyl]isoxazole-4-carboxamide, compound (XI) below, commonly known as leflunomide. Leflunomide is a medication of the DMARD type, used in active moderate to severe rheumatoid arthritis and psoriatic arthritis. It is a pyrimidine synthesis inhibitor.

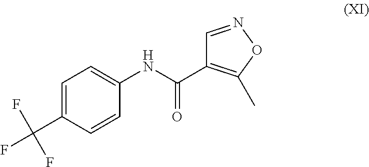

Analogs of leflunomide embodied herein include N-(phenyl)-3-carboxamidyl-5-methylisoxazole, N-(2-chlorophenyl)-3-carboxamidyl-5-methylisoxazole), N-(3-chlorophenyl)-3-carboxamidyl-5-methylisoxazole, N-(4-chlorophenyl)-3-carboxamidyl-5-methylisoxazole, N-(3-trifluoromethyl)-3-carboxamidyl-5-methylisoxazole, N-(4-trifluoromethylphenyl)-3-carboxamidyl-5-methylisoxazole, N-(2,4-dichlorophenyl)-3-carboxamidyl-5-methylisoxazole, N-(4-methoxyphenyl)-3-carboxamidyl-5-methylisoxazole, N-(3,4,5-trimethoxyphenyl)-3-carboxamidyl-5-methylisoxazole, 4-[(5-methylisoxazole-3-carbonyl)-amino]-benzoic acid, and N-(4-fluorophenyl)-3-carboxamidyl-5-methylisoxazole.

In another embodiment, the small molecule compound useful for any of the above embodiments is [R-(E)]-5,6-dihydro-4-methoxy-6-(2-phenylethenyl)-2H-pyran-2-one, compound (XII) below, commonly known as kawain. Kawain is a member of the class of kavalactones, which are the main psychoactive components of the roots of Piper methysticum (kava), a shrub common on some Pacific Ocean islands.

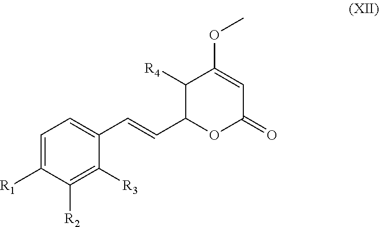

Kawain analogs embodied herein include yangonin, 10-methoxyyangonin, 11-methoxyyangonin, 11-hydroxyyangonin, 5,6-dehydrokavain, 11-methoxy-12-hydroxydehydrokavain, 7,8-dihydroyangonin, 5-hydroxykavain, 5,6-dihydroyangonin, 7,8-dihydrokavain, 5,6,7,8-tetrahydroyangonin, 5,6-dehydromethysticin, methysticin and 7,8-dihydromethysticin.

In another embodiment, the small molecule compound useful for any of the above embodiments is N-[3-(2,4-dichlorophenoxy)propyl]-N-methylprop-2-yn-1-amine, compound (XIII) below, commonly known as clorgyline. Clorgiline (or clorgyline) is an inhibitor of monoamine oxidase type A, structurally related to pargyline. It has antidepressant activity, and may potentially be useful in the treatment of Parkinson's disease.

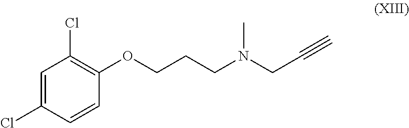

Analogs of clorgyline embodied herein include fluorochlorgyline and pargyline.

In another embodiment, the small molecule compound useful for any of the above embodiments is 3-hydroxy-13-methyl-9,11,12,14,15,16-hexahydro-6H-cylcopenta(a)phenanthren-17-one, compound (XIV) below, commonly known as equilin Equilin is one of the estrogens present in the mixture of estrogens isolated from horse urine and marketed as Premarin. Premarin became the most commonly used form of estrogen for hormone replacement therapy in the United States of America. Estrone is the major estrogen in Premarin (about 50%) and equilin is present as about 25% of the total.

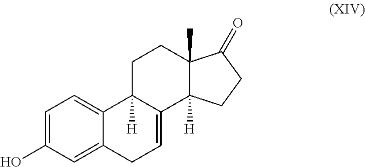

Analogs embodied herein include dihydroequilin, equilin sulfate, 4-hydroxy-equilin, 17-dihydroequilin 3-sulfate, 2-hydroxyequilin, 2-methoxyequilin and 4-methoxyequilin

Among the foregoing compounds and embodied herein are isomers thereof, prodrugs thereof, active metabolites thereof, and analogs thereof. “Active” means that the metabolite has biological activity in upregulating the utrophin promoter and/or increasing utrophin expression or activity.

In one embodiment, the term “isomer” includes, but is not limited to, optical isomers and analogs, structural isomers and analogs, conformational isomers and analogs, and the like.

In one embodiment, the term “prodrug” refers to an inactive pharmaceutical form of the compounds of this invention, and is metabolized in vivo into an active metabolite. In one embodiment, a prodrug of this invention includes, but not limited to a di-alkyl ester of a compound of this invention.

The invention includes “pharmaceutically acceptable salts” of the compounds of this invention, which may be produced, by reaction of a compound of this invention with an acid or base.

Suitable pharmaceutically-acceptable salts may be prepared from an inorganic acid or from an organic acid. In one embodiment, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In one embodiment, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoate, hydrofluorate, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, mitrates, naphthalenesulfonates, 2-naphthalenesulfonates, nicotinates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In one embodiment, examples of inorganic salts of carboxylic acids or phenols may be selected from ammonium, alkali metals to include lithium, sodium, potassium, cesium; alkaline earth metals to include calcium, magnesium, aluminium; zinc, barium, cholines, quaternary ammoniums.

In another embodiment, examples of organic salts of carboxylic acids or phenols may be selected from arginine, organic amines to include aliphatic organic amines, alicyclic organic amines, aromatic organic amines, benzathines, t-butylamines, benethamines (N-benzylphenethyl amine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In one embodiment, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

Pharmaceutically acceptable salts can be prepared from the amino groups, in other embodiments, by treatment with inorganic acids, for example, hydrochloric acid.

This invention provides, in some embodiments, derivatives of the above-mentioned compounds. In one embodiment, the term “derivatives” refers to amide derivatives, acid derivatives, ester derivatives or others, as known in the art. In another embodiment, this invention further includes hydrates of the compounds. In one embodiment, the term “hydrate” refers to hemihydrate, monohydrate, dihydrate, trihydrate or others, as known in the art.

This invention provides, in other embodiments, pharmaceutical products of the compounds of this invention. The term “pharmaceutical product” refers, in other embodiments, to a composition suitable for pharmaceutical use (pharmaceutical composition), for example, as described herein.

This invention provides, in other embodiments, metabolites of the above mentioned compounds. In one embodiment, the term “metabolite” refers to any substance produced from another substance by metabolism or a metabolic process.

In any of the embodiments herein, muscular dystrophy can be, by way of non-limiting example, Duchenne muscular dystrophy, Becker's muscular dystrophy, limb girdle muscular dystrophy, or a disease characterized by mutation or dysregulation of the dystrophin gene or an aberrant or dysfunctional gene product. In any of the embodiments herein, the subject can be a patient suffering from muscular dystrophy, such as but not limited to Duchenne muscular dystrophy, Becker's muscular dystrophy, limb girdle muscular dystrophy, or a disease characterized by mutation or dysregulation of the dystrophin gene or an aberrant or dysfunctional gene product.

Thus, in one embodiment, a method is provided for increasing activity of the utrophin promoter in a subject by administering to the subject a composition comprising a compound of formula of formula (I). In another embodiment of the foregoing method, a compound of formula (I) is administered parenterally or orally. In another embodiment, a method is provided for increasing expression or activity of utrophin in a subject by administering to the subject a composition comprising a compound of formula (I). In another embodiment of the foregoing method, a compound of formula of formula (I) administered parenterally or orally. In another embodiment, a method is provided for correcting a deficiency of dystrophin in a subject by administering to the subject a composition comprising a compound of formula (I). In another embodiment of the foregoing method, a compound of formula (I) administered parenterally or orally. In another embodiment, a method is provided for treating a subject with muscular dystrophy by administering a composition that increases activity of the utrophin promoter, the composition comprising a compound of formula (I). In another embodiment of the foregoing method, a compound of formula (I) administered parenterally or orally. In another embodiment, a method is provided for treating a subject with muscular dystrophy by administering a composition that increases expression or activity of utrophin, wherein the composition comprises a compound of formula (I). In another embodiment of the foregoing method, a compound of formula (I) administered parenterally or orally. In another embodiment, a method is provided for reducing the symptoms associated with muscular dystrophy in a subject comprising administering a composition that increases activity of the utrophin promoter, wherein the a compound of formula is a compound of formula (I). In another embodiment of the foregoing method, a compound of formula (I) administered parenterally or orally. In another embodiment, a method is provided for reducing the symptoms associated with muscular dystrophy in a subject comprising administering a composition that increases expression or activity of utrophin, wherein the a compound of formula is a compound of formula (I). In another embodiment of the foregoing method, a compound of formula (I) administered parenterally or orally.

In another embodiment, a method is provided for treating muscular dystrophy comprising administering a compound of formula (I). In another embodiment, a method is provided for reducing the symptoms of muscular dystrophy comprising administering a compound of formula (I).

In another embodiment, a method is provided for increasing activity of the utrophin promoter in a subject comprising the step of administering to the subject a composition comprising a compound of formula (I), whereby activity of the utrophin promoter is increased. In another embodiment of the foregoing method, a compound of formula (I) administered parenterally or orally. In one embodiment, a method is provided for increasing expression or activity of utrophin in a subject comprising the step of administering to the subject a composition comprising a compound of formula (I), wherein the expression or activity of utrophin is increased. In another embodiment of the foregoing method, a compound of formula (I) administered parenterally or orally. In another embodiment, a method is provided for correcting a deficiency of dystrophin in a subject comprising the step of by administering to the subject a composition comprising a compound of formula (I), wherein the deficiency of dystrophin is improved or corrected. In another embodiment of the foregoing method, a compound of formula (I) administered parenterally or orally. In another embodiment, a method is provided for treating a subject with muscular dystrophy comprising the step of administering a composition comprising a compound of formula (I) that increases activity of the utrophin promoter, wherein muscular dystrophy is treated. In another embodiment of the foregoing method, a compound of formula (I) is administered parenterally or orally. In another embodiment, a method is provided for treating a subject with muscular dystrophy comprising the step of administering a composition comprising a compound of formula (I) that increases expression or activity of utrophin, wherein muscular dystrophy is treated. In another embodiment of the foregoing method, a compound of formula (I) is administered parenterally or orally. In another embodiment, a method is provided for reducing the symptoms associated with muscular dystrophy in a subject comprising the step of administering a composition comprising a compound of formula (I) that increases activity of the utrophin promoter, wherein the symptoms of muscular dystrophy are reduced. In another embodiment of the foregoing method, a compound of formula (I) is administered parenterally or orally. In another embodiment, a method is provided for reducing the symptoms associated with muscular dystrophy in a subject comprising the step of administering a composition comprising a compound of formula (I) that increases expression or activity of utrophin, wherein the symptoms of muscular dystrophy are reduced. In another embodiment of the foregoing method, a compound of formula (I) is administered parenterally or orally.

In another embodiment, a method is provided for treating muscular dystrophy comprising the step of administering a composition comprising a compound of formula (I), wherein muscular dystrophy is treated. In another embodiment, a method is provided for reducing the symptoms of muscular dystrophy comprising the step of administering a composition comprising a compound of formula (I), wherein the symptoms of muscular dystrophy are reduced.

Thus, in one embodiment, a method is provided for increasing activity of the utrophin promoter in a subject by administering to the subject a composition comprising piperine, or an isomer, prodrug, active metabolite, analog, or a pharmaceutically-acceptable derivative or salt form of any of the foregoing. In another embodiment of the foregoing method, piperine, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising piperine, or an isomer, prodrug, active metabolite, analog, or a pharmaceutically-acceptable derivative or salt form of any of the foregoing. In another embodiment of the foregoing method, piperine, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising piperine, or an isomer, prodrug, active metabolite, analog, or a pharmaceutically-acceptable derivative or salt form of any of the foregoing. In another embodiment of the foregoing method, piperine, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising piperine, or an isomer, prodrug, active metabolite, analog, or a pharmaceutically-acceptable derivative or salt form of any of the foregoing. In another embodiment of the foregoing method, piperine, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises piperine, or an isomer, prodrug, active metabolite, analog, or a pharmaceutically-acceptable derivative or salt form of any of the foregoing. In another embodiment of the foregoing method, piperine, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is piperine, or an isomer, prodrug, active metabolite, analog, or a pharmaceutically-acceptable derivative or salt form of any of the foregoing. In another embodiment of the foregoing method, piperine, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is piperine, or an isomer, prodrug, active metabolite, analog, or a pharmaceutically-acceptable derivative or salt form of any of the foregoing. In another embodiment of the foregoing method, piperine, or an isomer, prodrug, active metabolite, analog, or a pharmaceutically-acceptable derivative or salt form of any of the foregoing administered parenterally or orally.

In another embodiment, a method is provided for treating muscular dystrophy comprising administering piperine, or an isomer, prodrug, active metabolite, analog, or a pharmaceutically-acceptable derivative or salt form of any of the foregoing. In another embodiment, a method is provided for reducing the symptoms of muscular dystrophy comprising administering piperine, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising piperine, or an isomer, prodrug, active metabolite, analog, or a pharmaceutically-acceptable derivative or salt form of any of the foregoing, whereby activity of the utrophin promoter is increased. In another embodiment of the foregoing method, piperine, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising piperine, or an isomer, prodrug, active metabolite, analog, or a pharmaceutically-acceptable derivative or salt form of any of the foregoing, wherein the expression or activity of utrophin is increased. In another embodiment of the foregoing method, piperine, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising piperine, or an isomer, prodrug, active metabolite, analog, or a pharmaceutically-acceptable derivative or salt form of any of the foregoing, wherein the deficiency of dystrophin is improved or corrected. In another embodiment of the foregoing method, piperine, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising piperine, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, piperine, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising piperine, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, piperine, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising piperine, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, piperine, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising piperine, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, piperine, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising piperine, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising piperine, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising apigenin, or an isomer, prodrug, active metabolite, analog, or a pharmaceutically-acceptable derivative or salt form of any of the foregoing. In another embodiment of the foregoing method, apigenin, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising apigenin, or an isomer, prodrug, active metabolite, analog, or a pharmaceutically-acceptable derivative or salt form of any of the foregoing. In another embodiment of the foregoing method, apigenin, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising apigenin, or an isomer, prodrug, active metabolite, analog, or a pharmaceutically-acceptable derivative or salt form of any of the foregoing. In another embodiment of the foregoing method, apigenin, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising apigenin, or an isomer, prodrug, active metabolite, analog, or a pharmaceutically-acceptable derivative or salt form of any of the foregoing. In another embodiment of the foregoing method, apigenin, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises apigenin, or an isomer, prodrug, active metabolite, analog, or a pharmaceutically-acceptable derivative or salt form of any of the foregoing. In another embodiment of the foregoing method, apigenin, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is apigenin, or an isomer, prodrug, active metabolite, analog, or a pharmaceutically-acceptable derivative or salt form of any of the foregoing. In another embodiment of the foregoing method, apigenin, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is apigenin, or an isomer, prodrug, active metabolite, analog, or a pharmaceutically-acceptable derivative or salt form of any of the foregoing. In another embodiment of the foregoing method, apigenin, or an isomer, prodrug, active metabolite, analog, or a pharmaceutically-acceptable derivative or salt form of any of the foregoing administered parenterally or orally.

In another embodiment, a method is provided for treating muscular dystrophy comprising administering apigenin, or an isomer, prodrug, active metabolite, analog, or a pharmaceutically-acceptable derivative or salt form of any of the foregoing. In another embodiment, a method is provided for reducing the symptoms of muscular dystrophy comprising administering apigenin, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising apigenin, or an isomer, prodrug, active metabolite, analog, or a pharmaceutically-acceptable derivative or salt form of any of the foregoing, whereby activity of the utrophin promoter is increased. In another embodiment of the foregoing method, apigenin, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising apigenin, or an isomer, prodrug, active metabolite, analog, or a pharmaceutically-acceptable derivative or salt form of any of the foregoing, wherein the expression or activity of utrophin is increased. In another embodiment of the foregoing method, apigenin, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising apigenin, or an isomer, prodrug, active metabolite, analog, or a pharmaceutically-acceptable derivative or salt form of any of the foregoing, wherein the deficiency of dystrophin is improved or corrected. In another embodiment of the foregoing method, apigenin, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising apigenin, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, apigenin, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising apigenin, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, apigenin, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising apigenin, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, apigenin, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising apigenin, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, apigenin, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising apigenin, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising apigenin, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising chrysin, or an isomer, prodrug, active metabolite, analog, or a pharmaceutically-acceptable derivative or salt form of any of the foregoing. In another embodiment of the foregoing method, chrysin, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising chrysin, or an isomer, prodrug, active metabolite, analog, or a pharmaceutically-acceptable derivative or salt form of any of the foregoing. In another embodiment of the foregoing method, chrysin, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising chrysin, or an isomer, prodrug, active metabolite, analog, or a pharmaceutically-acceptable derivative or salt form of any of the foregoing. In another embodiment of the foregoing method, chrysin, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising chrysin, or an isomer, prodrug, active metabolite, analog, or a pharmaceutically-acceptable derivative or salt form of any of the foregoing. In another embodiment of the foregoing method, chrysin, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises chrysin, or an isomer, prodrug, active metabolite, analog, or a pharmaceutically-acceptable derivative or salt form of any of the foregoing. In another embodiment of the foregoing method, chrysin, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is chrysin, or an isomer, prodrug, active metabolite, analog, or a pharmaceutically-acceptable derivative or salt form of any of the foregoing. In another embodiment of the foregoing method, chrysin, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is chrysin, or an isomer, prodrug, active metabolite, analog, or a pharmaceutically-acceptable derivative or salt form of any of the foregoing. In another embodiment of the foregoing method, chrysin, or an isomer, prodrug, active metabolite, analog, or a pharmaceutically-acceptable derivative or salt form of any of the foregoing administered parenterally or orally.

In another embodiment, a method is provided for treating muscular dystrophy comprising administering chrysin, or an isomer, prodrug, active metabolite, analog, or a pharmaceutically-acceptable derivative or salt form of any of the foregoing. In another embodiment, a method is provided for reducing the symptoms of muscular dystrophy comprising administering chrysin, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising chrysin, or an isomer, prodrug, active metabolite, analog, or a pharmaceutically-acceptable derivative or salt form of any of the foregoing, whereby activity of the utrophin promoter is increased. In another embodiment of the foregoing method, chrysin, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising chrysin, or an isomer, prodrug, active metabolite, analog, or a pharmaceutically-acceptable derivative or salt form of any of the foregoing, wherein the expression or activity of utrophin is increased. In another embodiment of the foregoing method, chrysin, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising chrysin, or an isomer, prodrug, active metabolite, analog, or a pharmaceutically-acceptable derivative or salt form of any of the foregoing, wherein the deficiency of dystrophin is improved or corrected. In another embodiment of the foregoing method, chrysin, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising chrysin, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, chrysin, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising chrysin, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, chrysin, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising chrysin, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, chrysin, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising chrysin, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, chrysin, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising chrysin, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising chrysin, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising nabumetone, or an isomer, prodrug, active metabolite, analog, or a pharmaceutically-acceptable derivative or salt form of any of the foregoing. In another embodiment of the foregoing method, nabumetone, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising nabumetone, or an isomer, prodrug, active metabolite, analog, or a pharmaceutically-acceptable derivative or salt form of any of the foregoing. In another embodiment of the foregoing method, nabumetone, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising nabumetone, or an isomer, prodrug, active metabolite, analog, or a pharmaceutically-acceptable derivative or salt form of any of the foregoing. In another embodiment of the foregoing method, nabumetone, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising nabumetone, or an isomer, prodrug, active metabolite, analog, or a pharmaceutically-acceptable derivative or salt form of any of the foregoing. In another embodiment of the foregoing method, nabumetone, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises nabumetone, or an isomer, prodrug, active metabolite, analog, or a pharmaceutically-acceptable derivative or salt form of any of the foregoing. In another embodiment of the foregoing method, nabumetone, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is nabumetone, or an isomer, prodrug, active metabolite, analog, or a pharmaceutically-acceptable derivative or salt form of any of the foregoing. In another embodiment of the foregoing method, nabumetone, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is nabumetone, or an isomer, prodrug, active metabolite, analog, or a pharmaceutically-acceptable derivative or salt form of any of the foregoing. In another embodiment of the foregoing method, nabumetone, or an isomer, prodrug, active metabolite, analog, or a pharmaceutically-acceptable derivative or salt form of any of the foregoing administered parenterally or orally.

In another embodiment, a method is provided for treating muscular dystrophy comprising administering nabumetone, or an isomer, prodrug, active metabolite, analog, or a pharmaceutically-acceptable derivative or salt form of any of the foregoing. In another embodiment, a method is provided for reducing the symptoms of muscular dystrophy comprising administering nabumetone, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising nabumetone, or an isomer, prodrug, active metabolite, analog, or a pharmaceutically-acceptable derivative or salt form of any of the foregoing, whereby activity of the utrophin promoter is increased. In another embodiment of the foregoing method, nabumetone, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising nabumetone, or an isomer, prodrug, active metabolite, analog, or a pharmaceutically-acceptable derivative or salt form of any of the foregoing, wherein the expression or activity of utrophin is increased. In another embodiment of the foregoing method, nabumetone, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising nabumetone, or an isomer, prodrug, active metabolite, analog, or a pharmaceutically-acceptable derivative or salt form of any of the foregoing, wherein the deficiency of dystrophin is improved or corrected. In another embodiment of the foregoing method, nabumetone, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising nabumetone, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, nabumetone, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising nabumetone, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, nabumetone, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising nabumetone, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, nabumetone, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising nabumetone, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, nabumetone, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising nabumetone, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising nabumetone, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising riluzole, or an isomer, prodrug, active metabolite, analog, or a pharmaceutically-acceptable derivative or salt form of any of the foregoing. In another embodiment of the foregoing method, riluzole, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising riluzole, or an isomer, prodrug, active metabolite, analog, or a pharmaceutically-acceptable derivative or salt form of any of the foregoing. In another embodiment of the foregoing method, riluzole, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising riluzole, or an isomer, prodrug, active metabolite, analog, or a pharmaceutically-acceptable derivative or salt form of any of the foregoing. In another embodiment of the foregoing method, riluzole, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising riluzole, or an isomer, prodrug, active metabolite, analog, or a pharmaceutically-acceptable derivative or salt form of any of the foregoing. In another embodiment of the foregoing method, riluzole, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises riluzole, or an isomer, prodrug, active metabolite, analog, or a pharmaceutically-acceptable derivative or salt form of any of the foregoing. In another embodiment of the foregoing method, riluzole, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is riluzole, or an isomer, prodrug, active metabolite, analog, or a pharmaceutically-acceptable derivative or salt form of any of the foregoing. In another embodiment of the foregoing method, riluzole, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is riluzole, or an isomer, prodrug, active metabolite, analog, or a pharmaceutically-acceptable derivative or salt form of any of the foregoing. In another embodiment of the foregoing method, riluzole, or an isomer, prodrug, active metabolite, analog, or a pharmaceutically-acceptable derivative or salt form of any of the foregoing administered parenterally or orally.

In another embodiment, a method is provided for treating muscular dystrophy comprising administering riluzole, or an isomer, prodrug, active metabolite, analog, or a pharmaceutically-acceptable derivative or salt form of any of the foregoing. In another embodiment, a method is provided for reducing the symptoms of muscular dystrophy comprising administering riluzole, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising riluzole, or an isomer, prodrug, active metabolite, analog, or a pharmaceutically-acceptable derivative or salt form of any of the foregoing, whereby activity of the utrophin promoter is increased. In another embodiment of the foregoing method, riluzole, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising riluzole, or an isomer, prodrug, active metabolite, analog, or a pharmaceutically-acceptable derivative or salt form of any of the foregoing, wherein the expression or activity of utrophin is increased. In another embodiment of the foregoing method, riluzole, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising riluzole, or an isomer, prodrug, active metabolite, analog, or a pharmaceutically-acceptable derivative or salt form of any of the foregoing, wherein the deficiency of dystrophin is improved or corrected. In another embodiment of the foregoing method, riluzole, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising riluzole, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, riluzole, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising riluzole, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, riluzole, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising riluzole, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, riluzole, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising riluzole, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, riluzole, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising riluzole, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising riluzole, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising hesperetin, or an isomer, prodrug, active metabolite, analog, or a pharmaceutically-acceptable derivative or salt form of any of the foregoing. In another embodiment of the foregoing method, hesperetin, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising hesperetin, or an isomer, prodrug, active metabolite, analog, or a pharmaceutically-acceptable derivative or salt form of any of the foregoing. In another embodiment of the foregoing method, hesperetin, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising hesperetin, or an isomer, prodrug, active metabolite, analog, or a pharmaceutically-acceptable derivative or salt form of any of the foregoing. In another embodiment of the foregoing method, hesperetin, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising hesperetin, or an isomer, prodrug, active metabolite, analog, or a pharmaceutically-acceptable derivative or salt form of any of the foregoing. In another embodiment of the foregoing method, hesperetin, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises hesperetin, or an isomer, prodrug, active metabolite, analog, or a pharmaceutically-acceptable derivative or salt form of any of the foregoing. In another embodiment of the foregoing method, hesperetin, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is hesperetin, or an isomer, prodrug, active metabolite, analog, or a pharmaceutically-acceptable derivative or salt form of any of the foregoing. In another embodiment of the foregoing method, hesperetin, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is hesperetin, or an isomer, prodrug, active metabolite, analog, or a pharmaceutically-acceptable derivative or salt form of any of the foregoing. In another embodiment of the foregoing method, hesperetin, or an isomer, prodrug, active metabolite, analog, or a pharmaceutically-acceptable derivative or salt form of any of the foregoing administered parenterally or orally.

In another embodiment, a method is provided for treating muscular dystrophy comprising administering hesperetin, or an isomer, prodrug, active metabolite, analog, or a pharmaceutically-acceptable derivative or salt form of any of the foregoing. In another embodiment, a method is provided for reducing the symptoms of muscular dystrophy comprising administering hesperetin, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising hesperetin, or an isomer, prodrug, active metabolite, analog, or a pharmaceutically-acceptable derivative or salt form of any of the foregoing, whereby activity of the utrophin promoter is increased. In another embodiment of the foregoing method, hesperetin, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising hesperetin, or an isomer, prodrug, active metabolite, analog, or a pharmaceutically-acceptable derivative or salt form of any of the foregoing, wherein the expression or activity of utrophin is increased. In another embodiment of the foregoing method, hesperetin, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising hesperetin, or an isomer, prodrug, active metabolite, analog, or a pharmaceutically-acceptable derivative or salt form of any of the foregoing, wherein the deficiency of dystrophin is improved or corrected. In another embodiment of the foregoing method, hesperetin, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising hesperetin, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, hesperetin, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising hesperetin, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, hesperetin, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising hesperetin, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, hesperetin, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising hesperetin, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, hesperetin, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising hesperetin, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising hesperetin, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising resveratrol, or an isomer, prodrug, active metabolite, analog, or a pharmaceutically-acceptable derivative or salt form of any of the foregoing. In another embodiment of the foregoing method, resveratrol, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising resveratrol, or an isomer, prodrug, active metabolite, analog, or a pharmaceutically-acceptable derivative or salt form of any of the foregoing. In another embodiment of the foregoing method, resveratrol, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising resveratrol, or an isomer, prodrug, active metabolite, analog, or a pharmaceutically-acceptable derivative or salt form of any of the foregoing. In another embodiment of the foregoing method, resveratrol, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising resveratrol, or an isomer, prodrug, active metabolite, analog, or a pharmaceutically-acceptable derivative or salt form of any of the foregoing. In another embodiment of the foregoing method, resveratrol, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises resveratrol, or an isomer, prodrug, active metabolite, analog, or a pharmaceutically-acceptable derivative or salt form of any of the foregoing. In another embodiment of the foregoing method, resveratrol, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is resveratrol, or an isomer, prodrug, active metabolite, analog, or a pharmaceutically-acceptable derivative or salt form of any of the foregoing. In another embodiment of the foregoing method, resveratrol, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is resveratrol, or an isomer, prodrug, active metabolite, analog, or a pharmaceutically-acceptable derivative or salt form of any of the foregoing. In another embodiment of the foregoing method, resveratrol, or an isomer, prodrug, active metabolite, analog, or a pharmaceutically-acceptable derivative or salt form of any of the foregoing administered parenterally or orally.

In another embodiment, a method is provided for treating muscular dystrophy comprising administering resveratrol, or an isomer, prodrug, active metabolite, analog, or a pharmaceutically-acceptable derivative or salt form of any of the foregoing. In another embodiment, a method is provided for reducing the symptoms of muscular dystrophy comprising administering resveratrol, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising resveratrol, or an isomer, prodrug, active metabolite, analog, or a pharmaceutically-acceptable derivative or salt form of any of the foregoing, whereby activity of the utrophin promoter is increased. In another embodiment of the foregoing method, resveratrol, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising resveratrol, or an isomer, prodrug, active metabolite, analog, or a pharmaceutically-acceptable derivative or salt form of any of the foregoing, wherein the expression or activity of utrophin is increased. In another embodiment of the foregoing method, resveratrol, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising resveratrol, or an isomer, prodrug, active metabolite, analog, or a pharmaceutically-acceptable derivative or salt form of any of the foregoing, wherein the deficiency of dystrophin is improved or corrected. In another embodiment of the foregoing method, resveratrol, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising resveratrol, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, resveratrol, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising resveratrol, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, resveratrol, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising resveratrol, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, resveratrol, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising resveratrol, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, resveratrol, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising resveratrol, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising resveratrol, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising phenazopyridine, or an isomer, prodrug, active metabolite, analog, or a pharmaceutically-acceptable derivative or salt form of any of the foregoing. In another embodiment of the foregoing method, phenazopyridine, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising phenazopyridine, or an isomer, prodrug, active metabolite, analog, or a pharmaceutically-acceptable derivative or salt form of any of the foregoing. In another embodiment of the foregoing method, phenazopyridine, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising phenazopyridine, or an isomer, prodrug, active metabolite, analog, or a pharmaceutically-acceptable derivative or salt form of any of the foregoing. In another embodiment of the foregoing method, phenazopyridine, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising phenazopyridine, or an isomer, prodrug, active metabolite, analog, or a pharmaceutically-acceptable derivative or salt form of any of the foregoing. In another embodiment of the foregoing method, phenazopyridine, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises phenazopyridine, or an isomer, prodrug, active metabolite, analog, or a pharmaceutically-acceptable derivative or salt form of any of the foregoing. In another embodiment of the foregoing method, phenazopyridine, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is phenazopyridine, or an isomer, prodrug, active metabolite, analog, or a pharmaceutically-acceptable derivative or salt form of any of the foregoing. In another embodiment of the foregoing method, phenazopyridine, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is phenazopyridine, or an isomer, prodrug, active metabolite, analog, or a pharmaceutically-acceptable derivative or salt form of any of the foregoing. In another embodiment of the foregoing method, phenazopyridine, or an isomer, prodrug, active metabolite, analog, or a pharmaceutically-acceptable derivative or salt form of any of the foregoing administered parenterally or orally.

In another embodiment, a method is provided for treating muscular dystrophy comprising administering phenazopyridine, or an isomer, prodrug, active metabolite, analog, or a pharmaceutically-acceptable derivative or salt form of any of the foregoing. In another embodiment, a method is provided for reducing the symptoms of muscular dystrophy comprising administering phenazopyridine, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising phenazopyridine, or an isomer, prodrug, active metabolite, analog, or a pharmaceutically-acceptable derivative or salt form of any of the foregoing, whereby activity of the utrophin promoter is increased. In another embodiment of the foregoing method, phenazopyridine, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising phenazopyridine, or an isomer, prodrug, active metabolite, analog, or a pharmaceutically-acceptable derivative or salt form of any of the foregoing, wherein the expression or activity of utrophin is increased. In another embodiment of the foregoing method, phenazopyridine, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising phenazopyridine, or an isomer, prodrug, active metabolite, analog, or a pharmaceutically-acceptable derivative or salt form of any of the foregoing, wherein the deficiency of dystrophin is improved or corrected. In another embodiment of the foregoing method, phenazopyridine, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising phenazopyridine, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, phenazopyridine, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising phenazopyridine, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, phenazopyridine, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising phenazopyridine, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, phenazopyridine, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising phenazopyridine, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, phenazopyridine, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising phenazopyridine, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising phenazopyridine, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising tiabendazole, or an isomer, prodrug, active metabolite, analog, or a pharmaceutically-acceptable derivative or salt form of any of the foregoing. In another embodiment of the foregoing method, tiabendazole, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising tiabendazole, or an isomer, prodrug, active metabolite, analog, or a pharmaceutically-acceptable derivative or salt form of any of the foregoing. In another embodiment of the foregoing method, tiabendazole, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising tiabendazole, or an isomer, prodrug, active metabolite, analog, or a pharmaceutically-acceptable derivative or salt form of any of the foregoing. In another embodiment of the foregoing method, tiabendazole, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising tiabendazole, or an isomer, prodrug, active metabolite, analog, or a pharmaceutically-acceptable derivative or salt form of any of the foregoing. In another embodiment of the foregoing method, tiabendazole, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises tiabendazole, or an isomer, prodrug, active metabolite, analog, or a pharmaceutically-acceptable derivative or salt form of any of the foregoing. In another embodiment of the foregoing method, tiabendazole, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is tiabendazole, or an isomer, prodrug, active metabolite, analog, or a pharmaceutically-acceptable derivative or salt form of any of the foregoing. In another embodiment of the foregoing method, tiabendazole, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is tiabendazole, or an isomer, prodrug, active metabolite, analog, or a pharmaceutically-acceptable derivative or salt form of any of the foregoing. In another embodiment of the foregoing method, tiabendazole, or an isomer, prodrug, active metabolite, analog, or a pharmaceutically-acceptable derivative or salt form of any of the foregoing administered parenterally or orally.

In another embodiment, a method is provided for treating muscular dystrophy comprising administering tiabendazole, or an isomer, prodrug, active metabolite, analog, or a pharmaceutically-acceptable derivative or salt form of any of the foregoing. In another embodiment, a method is provided for reducing the symptoms of muscular dystrophy comprising administering tiabendazole, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising tiabendazole, or an isomer, prodrug, active metabolite, analog, or a pharmaceutically-acceptable derivative or salt form of any of the foregoing, whereby activity of the utrophin promoter is increased. In another embodiment of the foregoing method, tiabendazole, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising tiabendazole, or an isomer, prodrug, active metabolite, analog, or a pharmaceutically-acceptable derivative or salt form of any of the foregoing, wherein the expression or activity of utrophin is increased. In another embodiment of the foregoing method, tiabendazole, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising tiabendazole, or an isomer, prodrug, active metabolite, analog, or a pharmaceutically-acceptable derivative or salt form of any of the foregoing, wherein the deficiency of dystrophin is improved or corrected. In another embodiment of the foregoing method, tiabendazole, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising tiabendazole, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, tiabendazole, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising tiabendazole, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, tiabendazole, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising tiabendazole, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, tiabendazole, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising tiabendazole, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, tiabendazole, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising tiabendazole, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising tiabendazole, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising leflunomide, or an isomer, prodrug, active metabolite, analog, or a pharmaceutically-acceptable derivative or salt form of any of the foregoing. In another embodiment of the foregoing method, leflunomide, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising leflunomide, or an isomer, prodrug, active metabolite, analog, or a pharmaceutically-acceptable derivative or salt form of any of the foregoing. In another embodiment of the foregoing method, leflunomide, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising leflunomide, or an isomer, prodrug, active metabolite, analog, or a pharmaceutically-acceptable derivative or salt form of any of the foregoing. In another embodiment of the foregoing method, leflunomide, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising leflunomide, or an isomer, prodrug, active metabolite, analog, or a pharmaceutically-acceptable derivative or salt form of any of the foregoing. In another embodiment of the foregoing method, leflunomide, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises leflunomide, or an isomer, prodrug, active metabolite, analog, or a pharmaceutically-acceptable derivative or salt form of any of the foregoing. In another embodiment of the foregoing method, leflunomide, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is leflunomide, or an isomer, prodrug, active metabolite, analog, or a pharmaceutically-acceptable derivative or salt form of any of the foregoing. In another embodiment of the foregoing method, leflunomide, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is leflunomide, or an isomer, prodrug, active metabolite, analog, or a pharmaceutically-acceptable derivative or salt form of any of the foregoing. In another embodiment of the foregoing method, leflunomide, or an isomer, prodrug, active metabolite, analog, or a pharmaceutically-acceptable derivative or salt form of any of the foregoing administered parenterally or orally.

In another embodiment, a method is provided for treating muscular dystrophy comprising administering leflunomide, or an isomer, prodrug, active metabolite, analog, or a pharmaceutically-acceptable derivative or salt form of any of the foregoing. In another embodiment, a method is provided for reducing the symptoms of muscular dystrophy comprising administering leflunomide, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising leflunomide, or an isomer, prodrug, active metabolite, analog, or a pharmaceutically-acceptable derivative or salt form of any of the foregoing, whereby activity of the utrophin promoter is increased. In another embodiment of the foregoing method, leflunomide, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising leflunomide, or an isomer, prodrug, active metabolite, analog, or a pharmaceutically-acceptable derivative or salt form of any of the foregoing, wherein the expression or activity of utrophin is increased. In another embodiment of the foregoing method, leflunomide, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising leflunomide, or an isomer, prodrug, active metabolite, analog, or a pharmaceutically-acceptable derivative or salt form of any of the foregoing, wherein the deficiency of dystrophin is improved or corrected. In another embodiment of the foregoing method, leflunomide, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising leflunomide, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, leflunomide, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising leflunomide, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, leflunomide, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising leflunomide, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, leflunomide, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising leflunomide, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, leflunomide, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising leflunomide, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising leflunomide, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising kawain, or an isomer, prodrug, active metabolite, analog, or a pharmaceutically-acceptable derivative or salt form of any of the foregoing. In another embodiment of the foregoing method, kawain, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising kawain, or an isomer, prodrug, active metabolite, analog, or a pharmaceutically-acceptable derivative or salt form of any of the foregoing. In another embodiment of the foregoing method, kawain, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising kawain, or an isomer, prodrug, active metabolite, analog, or a pharmaceutically-acceptable derivative or salt form of any of the foregoing. In another embodiment of the foregoing method, kawain, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising kawain, or an isomer, prodrug, active metabolite, analog, or a pharmaceutically-acceptable derivative or salt form of any of the foregoing. In another embodiment of the foregoing method, kawain, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises kawain, or an isomer, prodrug, active metabolite, analog, or a pharmaceutically-acceptable derivative or salt form of any of the foregoing. In another embodiment of the foregoing method, kawain, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is kawain, or an isomer, prodrug, active metabolite, analog, or a pharmaceutically-acceptable derivative or salt form of any of the foregoing. In another embodiment of the foregoing method, kawain, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is kawain, or an isomer, prodrug, active metabolite, analog, or a pharmaceutically-acceptable derivative or salt form of any of the foregoing. In another embodiment of the foregoing method, kawain, or an isomer, prodrug, active metabolite, analog, or a pharmaceutically-acceptable derivative or salt form of any of the foregoing administered parenterally or orally.

In another embodiment, a method is provided for treating muscular dystrophy comprising administering kawain, or an isomer, prodrug, active metabolite, analog, or a pharmaceutically-acceptable derivative or salt form of any of the foregoing. In another embodiment, a method is provided for reducing the symptoms of muscular dystrophy comprising administering kawain, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising kawain, or an isomer, prodrug, active metabolite, analog, or a pharmaceutically-acceptable derivative or salt form of any of the foregoing, whereby activity of the utrophin promoter is increased. In another embodiment of the foregoing method, kawain, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising kawain, or an isomer, prodrug, active metabolite, analog, or a pharmaceutically-acceptable derivative or salt form of any of the foregoing, wherein the expression or activity of utrophin is increased. In another embodiment of the foregoing method, kawain, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising kawain, or an isomer, prodrug, active metabolite, analog, or a pharmaceutically-acceptable derivative or salt form of any of the foregoing, wherein the deficiency of dystrophin is improved or corrected. In another embodiment of the foregoing method, kawain, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising kawain, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, kawain, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising kawain, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, kawain, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising kawain, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, kawain, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising kawain, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, kawain, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising kawain, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising kawain, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising clorgyline, or an isomer, prodrug, active metabolite, analog, or a pharmaceutically-acceptable derivative or salt form of any of the foregoing. In another embodiment of the foregoing method, clorgyline, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising clorgyline, or an isomer, prodrug, active metabolite, analog, or a pharmaceutically-acceptable derivative or salt form of any of the foregoing. In another embodiment of the foregoing method, clorgyline, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising clorgyline, or an isomer, prodrug, active metabolite, analog, or a pharmaceutically-acceptable derivative or salt form of any of the foregoing. In another embodiment of the foregoing method, clorgyline, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising clorgyline, or an isomer, prodrug, active metabolite, analog, or a pharmaceutically-acceptable derivative or salt form of any of the foregoing. In another embodiment of the foregoing method, clorgyline, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises clorgyline, or an isomer, prodrug, active metabolite, analog, or a pharmaceutically-acceptable derivative or salt form of any of the foregoing. In another embodiment of the foregoing method, clorgyline, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is clorgyline, or an isomer, prodrug, active metabolite, analog, or a pharmaceutically-acceptable derivative or salt form of any of the foregoing. In another embodiment of the foregoing method, clorgyline, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is clorgyline, or an isomer, prodrug, active metabolite, analog, or a pharmaceutically-acceptable derivative or salt form of any of the foregoing. In another embodiment of the foregoing method, clorgyline, or an isomer, prodrug, active metabolite, analog, or a pharmaceutically-acceptable derivative or salt form of any of the foregoing administered parenterally or orally.

In another embodiment, a method is provided for treating muscular dystrophy comprising administering clorgyline, or an isomer, prodrug, active metabolite, analog, or a pharmaceutically-acceptable derivative or salt form of any of the foregoing. In another embodiment, a method is provided for reducing the symptoms of muscular dystrophy comprising administering clorgyline, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising clorgyline, or an isomer, prodrug, active metabolite, analog, or a pharmaceutically-acceptable derivative or salt form of any of the foregoing, whereby activity of the utrophin promoter is increased. In another embodiment of the foregoing method, clorgyline, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising clorgyline, or an isomer, prodrug, active metabolite, analog, or a pharmaceutically-acceptable derivative or salt form of any of the foregoing, wherein the expression or activity of utrophin is increased. In another embodiment of the foregoing method, clorgyline, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising clorgyline, or an isomer, prodrug, active metabolite, analog, or a pharmaceutically-acceptable derivative or salt form of any of the foregoing, wherein the deficiency of dystrophin is improved or corrected. In another embodiment of the foregoing method, clorgyline, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising clorgyline, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, clorgyline, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising clorgyline, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, clorgyline, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising clorgyline, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, clorgyline, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising clorgyline, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, clorgyline, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising clorgyline, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising clorgyline, or an isomer, prodrug, active metabolite, analog, or a pharmaceutically-acceptable derivative or salt form of any of the foregoing, wherein the symptoms of muscular dystrophy are reduced.

Thus, in one embodiment, a method is provided for increasing activity of the utrophin promoter in a subject by administering to the subject a composition comprising equilin, or an isomer, prodrug, active metabolite, analog, or a pharmaceutically-acceptable derivative or salt form of any of the foregoing. In another embodiment of the foregoing method, equilin, or an isomer, prodrug, active metabolite, analog, or a pharmaceutically-acceptable derivative or salt form of any of the foregoing is administered parenterally or orally. In another embodiment, a method is provided for increasing expression or activity of utrophin in a subject by administering to the subject a composition comprising equilin, or an isomer, prodrug, active metabolite, analog, or a pharmaceutically-acceptable derivative or salt form of any of the foregoing. In another embodiment of the foregoing method, equilin, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject by administering to the subject a composition comprising equilin, or an isomer, prodrug, active metabolite, analog, or a pharmaceutically-acceptable derivative or salt form of any of the foregoing. In another embodiment of the foregoing method, equilin, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases activity of the utrophin promoter, the composition comprising equilin, or an isomer, prodrug, active metabolite, analog, or a pharmaceutically-acceptable derivative or salt form of any of the foregoing. In another embodiment of the foregoing method, equilin, or an isomer, prodrug, active metabolite, analog, or a pharmaceutically-acceptable derivative or salt form of any of the foregoing administered parenterally or orally. In another embodiment, a method is provided for treating a subject with muscular dystrophy by administering a composition that increases expression or activity of utrophin, wherein the composition comprises equilin, or an isomer, prodrug, active metabolite, analog, or a pharmaceutically-acceptable derivative or salt form of any of the foregoing. In another embodiment of the foregoing method, equilin, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases activity of the utrophin promoter, wherein the compound is equilin, or an isomer, prodrug, active metabolite, analog, or a pharmaceutically-acceptable derivative or salt form of any of the foregoing. In another embodiment of the foregoing method, equilin, or an isomer, prodrug, active metabolite, analog, or a pharmaceutically-acceptable derivative or salt form of any of the foregoing administered parenterally or orally. In another embodiment, a method is provided for reducing the symptoms associated with muscular dystrophy in a subject comprising administering a composition that increases expression or activity of utrophin, wherein the compound is equilin, or an isomer, prodrug, active metabolite, analog, or a pharmaceutically-acceptable derivative or salt form of any of the foregoing. In another embodiment of the foregoing method, equilin, or an isomer, prodrug, active metabolite, analog, or a pharmaceutically-acceptable derivative or salt form of any of the foregoing administered parenterally or orally.

In another embodiment, a method is provided for treating muscular dystrophy comprising administering equilin, or an isomer, prodrug, active metabolite, analog, or a pharmaceutically-acceptable derivative or salt form of any of the foregoing. In another embodiment, a method is provided for reducing the symptoms of muscular dystrophy comprising administering equilin, or an isomer, prodrug, active metabolite, analog, or a pharmaceutically-acceptable derivative or salt form of any of the foregoing.

In another embodiment, a method is provided for increasing activity of the utrophin promoter in a subject comprising the step of administering to the subject a composition comprising equilin, or an isomer, prodrug, active metabolite, analog, or a pharmaceutically-acceptable derivative or salt form of any of the foregoing, whereby activity of the utrophin promoter is increased. In another embodiment of the foregoing method, equilin, or an isomer, prodrug, active metabolite, analog, or a pharmaceutically-acceptable derivative or salt form of any of the foregoing administered parenterally or orally. In one embodiment, a method is provided for increasing expression or activity of utrophin in a subject comprising the step of administering to the subject a composition comprising equilin, or an isomer, prodrug, active metabolite, analog, or a pharmaceutically-acceptable derivative or salt form of any of the foregoing, wherein the expression or activity of utrophin is increased. In another embodiment of the foregoing method, equilin, or an isomer, prodrug, active metabolite, analog, or a pharmaceutically-acceptable derivative or salt form of any of the foregoing administered parenterally or orally. In another embodiment, a method is provided for correcting a deficiency of dystrophin in a subject comprising the step of by administering to the subject a composition comprising equilin, or an isomer, prodrug, active metabolite, analog, or a pharmaceutically-acceptable derivative or salt form of any of the foregoing, wherein the deficiency of dystrophin is improved or corrected. In another embodiment of the foregoing method, equilin, or an isomer, prodrug, active metabolite, analog, or a pharmaceutically-acceptable derivative or salt form of any of the foregoing administered parenterally or orally. In another embodiment, a method is provided for treating a subject with muscular dystrophy comprising the step of administering a composition comprising equilin, or an isomer, prodrug, active metabolite, analog, or a pharmaceutically-acceptable derivative or salt form of any of the foregoing that increases activity of the utrophin promoter, wherein muscular dystrophy is treated. In another embodiment of the foregoing method, equilin, or an isomer, prodrug, active metabolite, analog, or a pharmaceutically-acceptable derivative or salt form of any of the foregoing is administered parenterally or orally. In another embodiment, a method is provided for treating a subject with muscular dystrophy comprising the step of by administering a composition comprising equilin, or an isomer, prodrug, active metabolite, analog, or a pharmaceutically-acceptable derivative or salt form of any of the foregoing that increases expression or activity of utrophin, wherein muscular dystrophy is treated. In another embodiment of the foregoing method, equilin, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising equilin, or an isomer, prodrug, active metabolite, analog, or a pharmaceutically-acceptable derivative or salt form of any of the foregoing that increases activity of the utrophin promoter, wherein the symptoms of muscular dystrophy are reduced. In another embodiment of the foregoing method, equilin, or an isomer, prodrug, active metabolite, analog, or a pharmaceutically-acceptable derivative or salt form of any of the foregoing is administered parenterally or orally. In another embodiment, a method is provided for reducing the symptoms associated with muscular dystrophy in a subject comprising the step of administering a composition comprising equilin, or an isomer, prodrug, active metabolite, analog, or a pharmaceutically-acceptable derivative or salt form of any of the foregoing that increases expression or activity of utrophin, wherein the symptoms of muscular dystrophy are reduced. In another embodiment of the foregoing method, equilin, or an isomer, prodrug, active metabolite, analog, or a pharmaceutically-acceptable derivative or salt form of any of the foregoing is administered parenterally or orally.

In another embodiment, a method is provided for treating muscular dystrophy comprising the step of administering a composition comprising equilin, or an isomer, prodrug, active metabolite, analog, or a pharmaceutically-acceptable derivative or salt form of any of the foregoing, wherein muscular dystrophy is treated. In another embodiment, a method is provided for reducing the symptoms of muscular dystrophy comprising the step of administering a composition comprising equilin, or an isomer, prodrug, active metabolite, analog, or a pharmaceutically-acceptable derivative or salt form of any of the foregoing, wherein the symptoms of muscular dystrophy are reduced.

### Pharmaceutical Compositions and Methods of Administration

The compounds of the present invention and pharmaceutical compositions comprising same can be, in another embodiment, administered to a subject by any method known to a person skilled in the art, such as orally, parenterally, paracancerally, transmucosally, transdermally, intramuscularly, intravenously, intradermally, subcutaneously, intraperitoneally, intra-ventricularly, intracranially, intravaginally or intratumorally. Typically oral or subcutaneous administration is used.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound (e.g. the mimetic compound, peptide or nucleotide molecule) and the inert carrier or diluent, a hard gelatin capsule.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Topical formulations include, in another embodiment, gels, ointments, creams, lotions, drops and the like.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active agent over a period of time.

In another embodiment, the active compound is delivered in a vesicle, e.g. a liposome.

In other embodiments, carriers or diluents used in methods of the present invention include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In another embodiment, parenteral vehicles (for subcutaneous, intravenous, intra-arterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In other embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention.

In another embodiment, the pharmaceutical compositions provided herein are controlled-release compositions, i.e. compositions in which the active compound is released over a period of time after administration. Controlled- or sustained-release compositions include formulation in lipophilic depots (e.g. fatty acids, waxes, oils). In another embodiment, the composition is an immediate-release composition, i.e. a composition in which of the active compound is released immediately after administration.

In another embodiment, the pharmaceutical composition is delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant. In yet another embodiment, a controlled release system is placed in proximity to the target cell, thus requiring only a fraction of the systemic dose (see, e.g., Goodson, in Medical Applications of Controlled Release, supra, vol. 2, pp. 115-138 (1984); and Langer R, Science 249: 1527-1533 (1990).

The compositions also include, in another embodiment, incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also included in the present invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds. Such modifications may also increase the compounds solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

Each of the above additives, excipients, formulations and methods of administration represents a separate embodiment of the present invention.

In one embodiment, the methods of the present invention comprise administering an active compound as the sole active ingredient. However, also encompassed within the scope of the present invention are methods for treating diseases and disorders that comprise administering the active compound in combination with one or more therapeutic agents. These agents include, but are not limited to, insulin agents, immunosuppressive agents, or drugs treating MS. In another embodiment, these agents are appropriate for the disease or disorder that is being treated, as is well known in the art.

In one embodiment, the methods of the present invention comprise administering an active compound as the sole active ingredient. However, also encompassed within the scope of the present invention are methods for treating diseases and disorders that comprise administering the active compound in combination with one or more therapeutic agents. In another embodiment, these agents are appropriate for the disease or disorder that is being treated, as is well known in the art.

### EXAMPLES

The ability of test compounds to upregulate activity of the utrophin promoter was determined using the muscle cell line C2C12utrn, which contains the utrophin gene promoter linked to the protein-encoding sequence for luciferase. Using these cells, a chemical library was screened for compounds that activate the utrophin promoter. Light production was the readout.

In the first screening, 384-well plates were used to grow C2C12 μm cells and expose to test compounds. Cells were exposed to test compounds at a single concentration for 24 hours, wherein 19 compounds of 1120 tested produced an increase of at least 20% of the activity of the luciferase reporter.

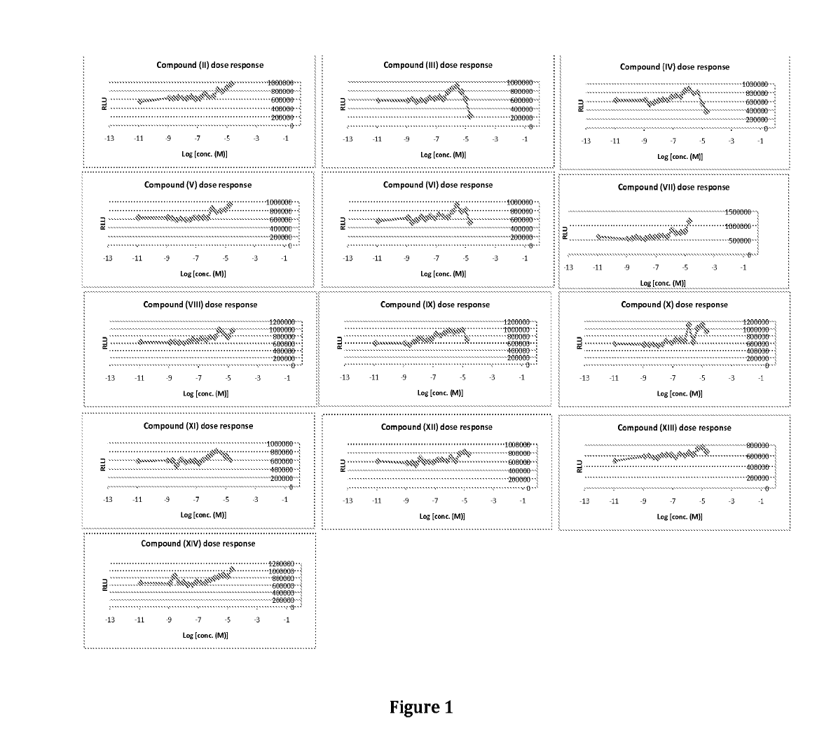

Subsequently, the 19 compounds were rescreened at 15 different concentrations. The results of the assay are shown in FIG. 1. Fourteen of the 19 compounds showed a dose-dependent activation of the utrophin promoter.

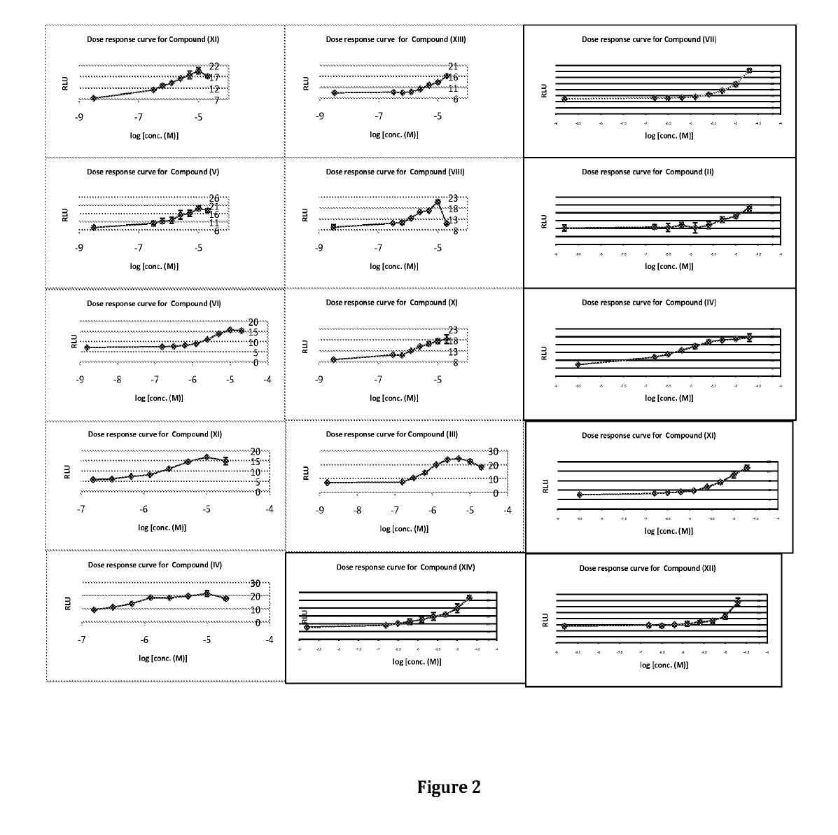

In some cases compounds were tested at a range of concentrations on test cells in a 96-well plate format. These results are shown in FIG. 2. Dose-dependent activation of the utrophin promoter was confirmed in 13 of the 14 compounds.

Thirteen compounds were found to exhibit dose-dependent activation of the utrophin promoter: The compounds and their codes shown in FIGS. 1 and 2 identified are: piperine [compound (II)]; apigenin [compound (III)]; chrysin [compound (IV)]; nabumetone [compound V)]; riluzole HCl [compound (VI)]; hesperetin [compound (VII)]; resveratrol [compound (VIII)]; phenazopyridine HCl [compound (IX)]; tiabendazole [compound (X)]; leflunomide [compound (XI)]; kawain [compound (XII)]; clorgyline HCl [compound (XIII)]; and equilin [compound (XIV)].

Moreover, the data from the screens on these compounds is summarized in the following table:

| | | 384-well | | | |
|---|---|---|---|---|---|
| | | format | | 96-well format | |
| | | Max. | Optimal | Max. | |
| | | fold | dose | fold | Optimal |
| Compound | Compound | change | (μM) | change | dose (μM) |
| hesperetin | (VII) | 1.94 | 20 | 2.9 | 20 |
| tiabendazole | (X) | 1.86 | 10 | 2.1 | 20 |
| resveratrol | (VIII) | 1.62 | 2.5 | 2.2 | 10 |
| phenazopyridine | (IX) | 1.62 | 10 | 2.6 | 10 |
| HCl | | | | | |
| equilin | (XIV) | 1.60 | 20 | 3.4 | 20 |
| piperine | (II) | 1.59 | 20 | 2.3 | 20 |
| nabumetone | (V) | 1.56 | 20 | 2.5 | 10 |
| riluzole HCl | (VI) | 1.56 | 2.5 | 2.2 | 10 |
| apigenin | (III) | 1.54 | 2.5 | 3.5 | 5 |
| chrysin | (IV) | 1.50 | 1.25 | 3.4 | 20 |
| leflunomide | (XI) | 1.38 | 2.5 | 2.9 | 20 |
| kawain | (XII) | 1.37 | 10 | 2.5 | 20 |
| clorgyline HCl | (XIII) | 1.16 | 10 | 1.9 | 20 |

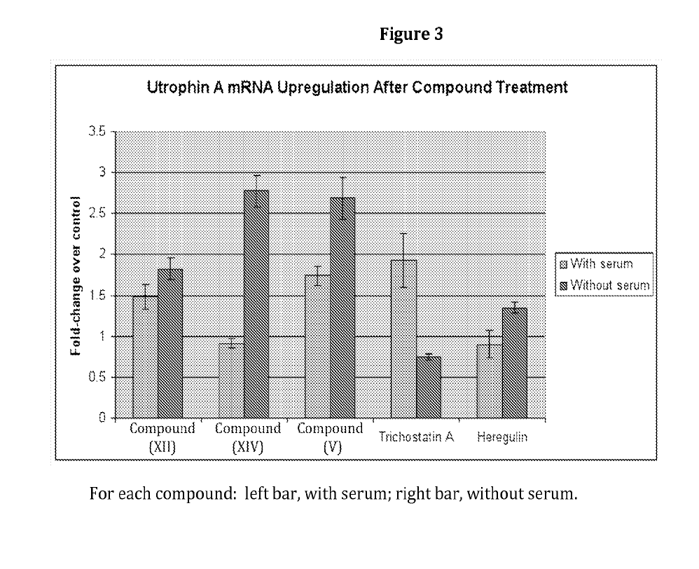

In a further experiment, the activity of kawain, equilin and nabumetone were evaluated in C2C12 muscle cells for upregulation of utrophin A mRNA (FIG. 3). C2C12 muscle cells were treated with optimal doses of compounds (based on previous dose-response data) for 24 hours, either in the presence of serum or in the absence of serum (following 24 hours of serum starvation). Trichostatin A and heregulin were used as positive controls for utrophin upregulation in the presence (trichostatin A) or absence (heregulin) of serum. Utrophin A mRNA was measured using a TaqMan qPCR assay and normalised to 18S rRNA. Data are presented as fold-changes in utrophin A mRNA compared to DMS O-only controls (compounds) or media-only controls (trichostatin A and heregulin).

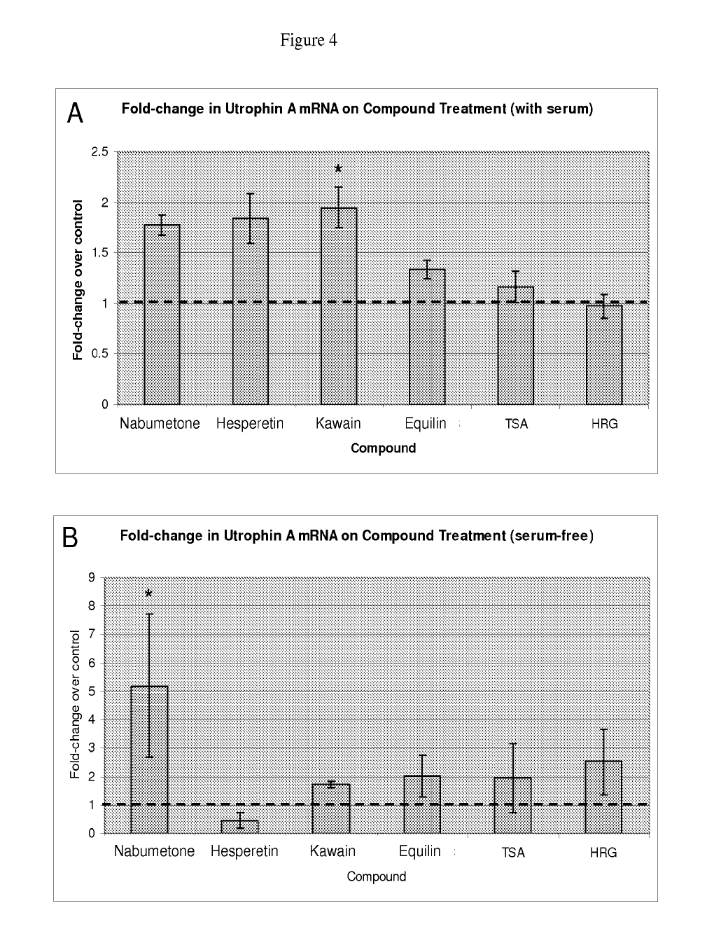

Additional experiments were performed in C2C12 cells. As shown in FIG. 4, the compounds increase endogenous Utrophin A mRNA. C2C12 cells were treated with Prestwick compounds A, J, L and S plus TSA and heregulin (HRG) as positive controls. All compounds increased levels of endogenous utrophin A mRNA either in serum-containing or serum-free media.

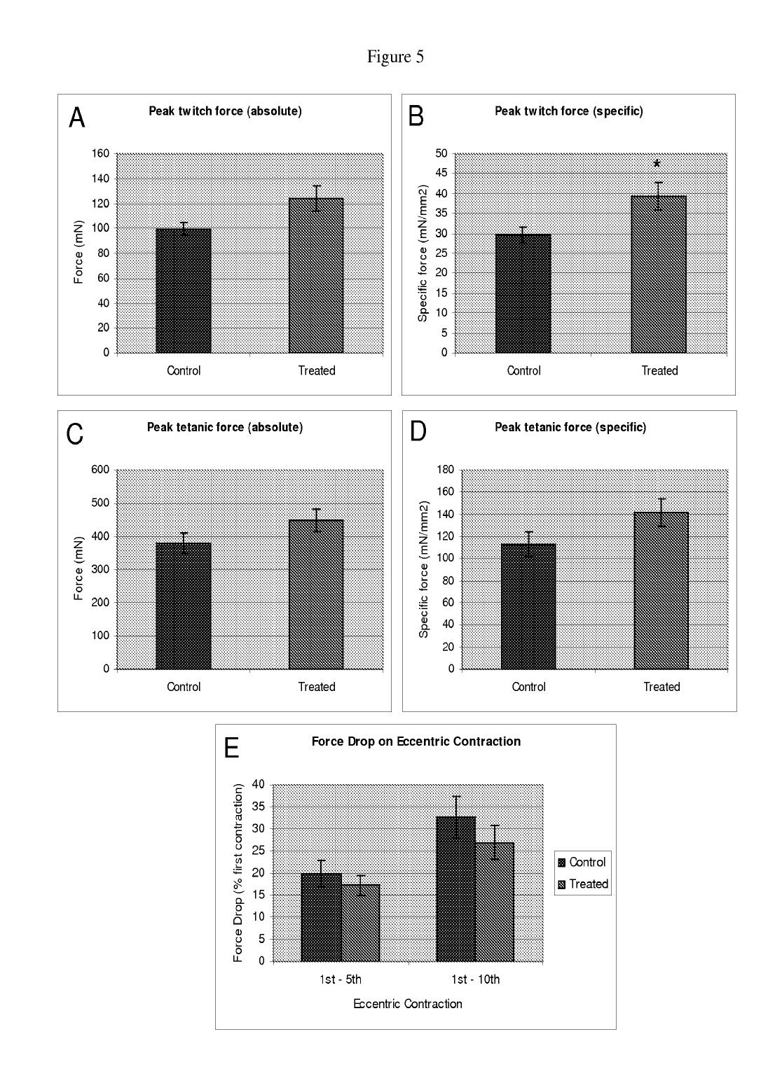

In vivo experiments were perfomed in mice by treating with nabumetone. As shown in FIG. 5, muscle weights were increased on treatment. Soleus, gastrocnemius (gastroc), tibialis anterior (TA) and quadriceps (quads) were bigger in treated mice. EDL was not significantly bigger (* p<0.05). There was no difference in whole body weight between treatment groups.

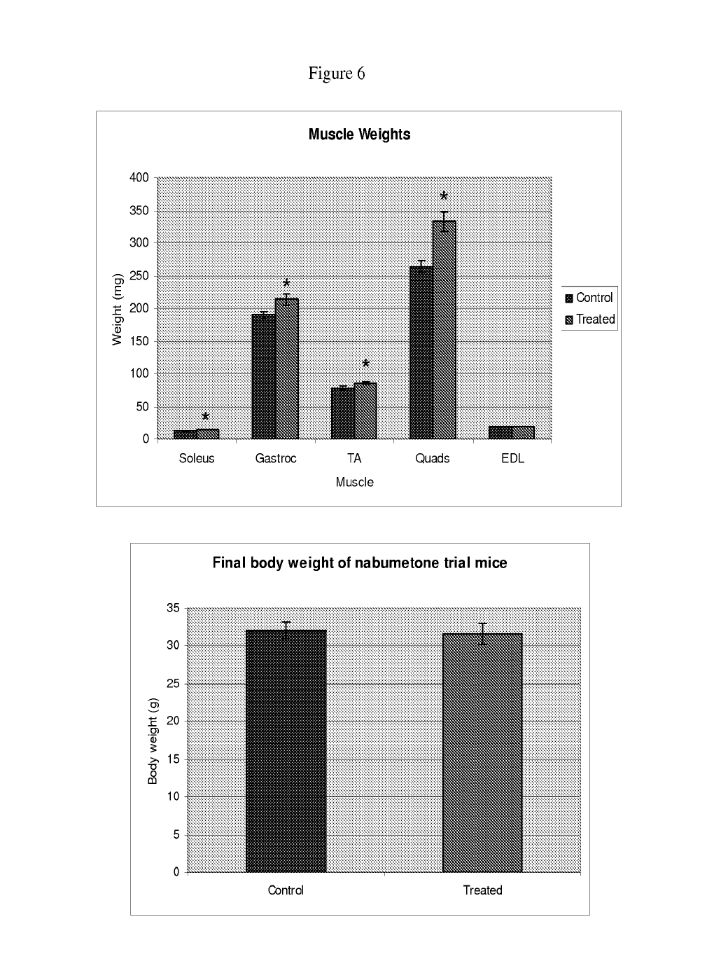

FIG. 6 shows that treated muscles show increased force generation. Muscle function tested using a variety of parameters showed a trend towards increased force generating capacity and decreased force drop on eccentric contraction. Peak specific twitch force was significantly increased (* p<0.05).

Thus, the aforementioned compounds and their isomers, metabolites, prodrugs and analogs embodied herein, by increasing activity of the utrophin promoter and thus increasing expression or activity of utrophin, which corrects the deficiency in dystrophin expression or activity in muscular dystrophy, have potential therapeutic utility in this disease.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method for treating and/or reducing the symptoms of muscular dystrophy in a subject by administering an effective amount of a composition comprising a compound selected from the group consisting of piperine, apigenin, chrysin, nabumetone, riluzole, hesperetin, resveratrol, phenazopyridine, tiabendazole, leflunomide, kawain, clorgyline, equilin; and an isomer, prodrug, active metabolite, analog, or a pharmaceutically-acceptable derivative or salt form of any of the foregoing, wherein the muscular dystrophy is treated and/or the symptoms of the muscular dystrophy are reduced.

2. A method for treating and/or reducing the symptoms of muscular dystrophy in a subject by administering an effective amount of a composition comprising a compound of formula (I): 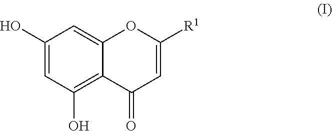
wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing,
wherein the muscular dystrophy is treated and/or the symptoms of the muscular dystrophy are reduced.

3.-8. (canceled)

9. A method for correcting a deficiency of dystrophin in a subject by administering to the subject an effective amount of a composition comprising a compound selected from the group consisting of piperine, apigenin, chrysin, nabumetone, riluzole, hesperetin, resveratrol, phenazopyridine, tiabendazole, leflunomide, kawain, clorgyline, equilin; and an isomer, prodrug, active metabolite, analog, or a pharmaceutically-acceptable derivative or salt form of any of the foregoing, wherein a deficiency of dystrophin is corrected.

10. A method for correcting a deficiency of dystrophin in a subject by administering to the subject an effective amount of a composition comprising a compound of formula (I): 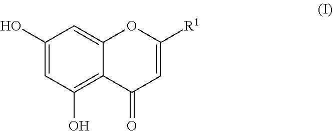
wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing,
wherein a deficiency of dystrophin is corrected.

11. A method for treating and/or reducing the symptoms of a subject with muscular dystrophy by administering an effective amount of composition comprising a utrophin promoter upregulator that increases activity of the utrophin promoter and/or increases expression or activity of utrophin, wherein activity of the utrophin promoter is increased and/or expression or activity of utrophin is increased, wherein muscular dystrophy is treated and/or the symptoms of the muscular dystrophy are reduced.

12. The method of claim 11 wherein the utrophin promoter upregulator is a compound selected from the group consisting of piperine, apigenin, chrysin, nabumetone, riluzole, hesperetin, resveratrol, phenazopyridine, tiabendazole, leflunomide, kawain, clorgyline, equilin; and an isomer, prodrug, active metabolite, analog, or a pharmaceutically-acceptable derivative or salt form of any of the foregoing.

13. The method of claim 11 wherein the utrophin promoter upregulator is a compound of formula (I): 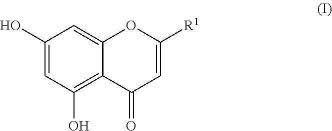
wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

14.-44. (canceled)

45. A method for upregulating utrophin expression or activity in a cell comprising exposing the cell to an effective amount of a compound selected from among piperine, apigenin, chrysin, nabumetone, riluzole, hesperetin, resveratrol, phenazopyridine, tiabendazole, leflunomide, kawain, clorgyline, equilin; or an isomer, prodrug, active metabolite, analog, or a pharmaceutically-acceptable derivative or salt form of any of the foregoing.

46. A method for upregulating utrophin expression or activity in a cell comprising exposing the cell to an effective amount of a compound of formula (I): 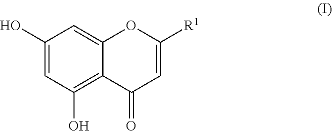
wherein R¹ is H, halogen, cyano, alkyloxy, nitro, NH₂, NHCOR², NHSO₂R², CONHR², COOR², optionally substituted alkyl, aryl, heteroalkyl, or heteroaryl; and
R² is H, hydroxy, halogen, cyano, alkyloxy, nitro, NH₂, or an optionally substituted alkyl, aryl, heteroalkyl, or heteroaryl;
or an isomer, prodrug, active metabolite, analog, or a pharmaceutically-acceptable derivative or salt form of any of the foregoing.

47. The method of claim 1 wherein the muscular dystrophy is Duchenne muscular dystrophy, Becker's muscular dystrophy, limb girdle muscular dystrophy, or a disease characterized by mutation or dysregulation of the dystrophin gene or an aberrant or dysfunctional dystrophin gene product.

48. The method of claim 9 wherein the subject is suffering from muscular dystrophy.

49. The method of claim 48 wherein the muscular dystrophy is Duchenne muscular dystrophy, Becker's muscular dystrophy, limb girdle muscular dystrophy, or a disease characterized by mutation or dysregulation of the dystrophin gene or an aberrant or dysfunctional dystrophin gene product.

50. The method of claim 2 wherein the muscular dystrophy is Duchenne muscular dystrophy, Becker's muscular dystrophy, limb girdle muscular dystrophy, or a disease characterized by mutation or dysregulation of the dystrophin gene or an aberrant or dysfunctional dystrophin gene product.

51. The method of claim 11 wherein the muscular dystrophy is Duchenne muscular dystrophy, Becker's muscular dystrophy, limb girdle muscular dystrophy, or a disease characterized by mutation or dysregulation of the dystrophin gene or an aberrant or dysfunctional dystrophin gene product.

52. The method of claim 10 wherein the subject is suffering from muscular dystrophy, and wherein the muscular dystrophy is Duchenne muscular dystrophy, Becker's muscular dystrophy, limb girdle muscular dystrophy, or a disease characterized by mutation or dysregulation of the dystrophin gene or an aberrant or dysfunctional dystrophin gene product.
